(19) United States
(12) Patent Application Publication
(10) Pub. No.: US 2011/0270358 A1
(43) Pub. Date: Nov. 3, 2011

(54) IMPLANTABLE MEDICAL DEVICE PROGRAMMING USING GESTURE-BASED CONTROL

(75) Inventors: Jon P. Davis, St. Michael, MN (US); Dennis J. Bourget, St. Michael, MN (US); Brent A. Huhta, Big Lake, MN (US); Rajeev M. Sahasrabudhe, Maple Grove, MN (US)

(73) Assignee: Medtronic, Inc., Minneapolis, MN (US)

(21) Appl. No.: 13/073,562

(22) Filed: Mar. 28, 2011

Related U.S. Application Data
(60) Provisional application No. 61/330,160, filed on Apr. 30, 2010.

(51) Int. Cl. G06F 3/048; G06F 3/16; A61N 1/36

(52) U.S. Cl. 607/60; 715/846; 715/727

(57) ABSTRACT

A programming device includes a touchscreen display, a processor, and a communication module. The processor controls the display to present a graphical icon on a first portion of the display. The processor detects a gesture-based contact between an object and the first portion of the display and determines a value of a therapy parameter associated with therapy delivered by a medical device based on the detection of the gesture-based contact. The communication module transmits information to the medical device to control the medical device to deliver the therapy based on the value of the therapy parameter.

This application claims the benefit of U.S. Provisional Application No. 61/330,160 by Davis et al., entitled, “IMPLANTABLE MEDICAL DEVICE PROGRAMMING USING GESTURE-BASED CONTROL” and filed on Apr. 30, 2010, the entire content of which is incorporated herein by reference.

### TECHNICAL FIELD

The disclosure relates to adjusting parameters for an implantable medical device, and more particularly, to adjusting parameters using a programmer that includes a touchscreen.

### BACKGROUND

Implantable electrical stimulators may be used to deliver electrical stimulation therapy to patients to treat a variety of symptoms or conditions such as chronic pain, tremor, Parkinson's disease, epilepsy, urinary or fecal incontinence, sexual dysfunction, obesity, or gastroparesis. In general, an implantable stimulator may deliver stimulation therapy (e.g., neurostimulation therapy) in the form of electrical pulses or continuous waveforms. An implantable stimulator may deliver stimulation therapy via one or more leads that include electrodes located proximate to target locations associated with the brain, the spinal cord, pelvic nerves, peripheral nerves, or the gastrointestinal tract of a patient. Hence, stimulation may be used in different therapeutic applications, such as deep brain stimulation (DBS), spinal cord stimulation (SCS), pelvic stimulation, gastric stimulation, or peripheral nerve stimulation. Stimulation also may be used for muscle stimulation, e.g., functional electrical stimulation (FES), to promote muscle movement or prevent atrophy.

In general, a clinician selects values for a number of stimulation parameters in order to define the electrical stimulation therapy to be delivered by the implantable stimulator. For example, the clinician may select stimulation parameters that define a current or voltage amplitude of electrical pulses delivered by the stimulator, a pulse rate, a pulse width, and a configuration of electrodes that deliver the pulses, e.g., in terms of selected electrodes and associated polarities. The stimulation parameters selected by the clinician may be referred to as a “stimulation program.” In some cases, therapy corresponding to multiple programs may be delivered on an alternating or continuous basis, as a group of programs.

The process of selecting the stimulation parameters may be done through trial and error before an efficacious stimulation program is discovered. An efficacious stimulation program may be a program that best balances greater clinical efficacy and minimal side effects experienced by the patient. The clinician may determine a most efficacious stimulation program by recording notes on the efficacy and side effects of each combination of stimulation parameters after delivery of stimulation via that combination. In some cases, efficacy and side effects of the stimulation parameters can be observed immediately. For example, SCS may produce paresthesia and side effects that can be observed by the clinician based on immediate patient feedback. Accordingly, the clinician may able to select the most efficacious stimulation program based on immediate receipt of patient feedback and/or observation of symptoms.

### SUMMARY

The disclosure is directed to techniques for gesture-based control of a medical device, such as an implantable medical device (IMD) that delivers therapy to a patient. In some examples, the IMD may be an implantable electrical stimulator that delivers electrical stimulation therapy, such as neurostimulation therapy. The techniques may be peformed using a programmer that communicates with the medical device. The programmer may include a touchscreen display that presents a graphical, gesture-based input medium, such as a graphical scroll wheel. A user may apply gestures to the gesture-based input medium to adjust one or more medical device parameters.

In one example, the disclosure provides a programming device that comprises a touchscreen display, a processor, and a communication module. The processor controls the display to present a graphical icon on a first portion of the display. The processor detects a gesture-based contact between an object and the first portion of the display and determines a value of a therapy parameter associated with therapy delivered by a medical device based on the detection of the gesture-based contact. The communication module transmits information to the medical device to control the medical device to deliver the therapy based on the value of the therapy parameter.

In another example, the disclosure provides a method that comprises presenting a graphical icon on a first portion of a touchscreen display and detecting a gesture-based contact between an object and the first portion of the display. The method further comprises determining a value of a therapy parameter associated with therapy delivered by a medical device based on the detection of the gesture-based contact. Additionally, the method comprises transmitting information to the medical device to control the medical device to deliver the therapy based on the value of the therapy parameter.

The details of one or more embodiments of the disclosure are set forth in the accompanying drawings and the description below. Other features, objects, and advantages of the disclosure will be apparent from the description and drawings, and from the claims.

### BRIEF DESCRIPTION OF THE DRAWINGS

FIG. 1 is a conceptual diagram of an implantable electrical stimulator and an associated programmer according to an example of the present disclosure.

FIG. 2 is a functional block diagram of the implantable electrical stimulator.

FIG. 3 is a functional block diagram of the programmer according to an example of the present disclosure.

FIG. 4 is a conceptual illustration of a graphical user interface (GUI) that facilitates programming of the implantable electrical stimulator using a graphical gesture-based input medium in the form of a scroll wheel, according to an example of the present disclosure.

FIG. 5 is another conceptual illustration of a GUI that facilitates programming of the implantable electrical stimulator using a graphical gesture-based input medium in the form of a scroll wheel, according to an example of the present disclosure.

FIG. 6 is a conceptual illustration of a GUI that facilitates adjusting a stimulation field of the implantable electrical stimulator using a graphical gesture-based input medium in the form of a scroll wheel according to an example of the present disclosure.

FIG. 7 is a conceptual illustration of a GUI that facilitates programming of the implantable electrical stimulator using a horizontal scroll wheel according to an example of the present disclosure.

FIG. 8 is a conceptual illustration of a GUI that facilitates programming of the implantable electrical stimulator using two scroll wheels according to an example of the present disclosure.

FIG. 9 is a conceptual illustration of a GUI that facilitates programming of the implantable electrical stimulator using a control wheel according to an example of the present disclosure.

FIG. 10 is a conceptual illustration of a GUI that facilitates programming of the implantable electrical stimulator using an omni-directional control according to an example of the present disclosure.

FIG. 11A is a conceptual illustration of a cathodal control shape according to an example of the present disclosure.

FIG. 11B is a conceptual illustration of a modification to the cathodal control shape of FIG. 11A according to an example of the present disclosure.

FIG. 12A is a conceptual diagram that illustrates an internal shape that indicates the amplitude associated with electrodes of a cathodal control shape according to an example of the present disclosure.

FIG. 12B is a conceptual diagram that illustrates a modification to the internal shape of FIG. 12A according to an example of the present disclosure.

FIG. 13 is a conceptual illustration of a GUI that facilitates mapping of paresthesia/pain felt by a patient according to an example of the present disclosure.

FIG. 14 is a conceptual illustration of a GUI that facilitates panning and zooming to view representations of implanted electrodes according to an example of the present disclosure.

FIG. 15 is a conceptual illustration of a GUI that facilitates programming of the implantable electrical stimulator using a scroll wheel that is positioned adjacent to a bezel of a display according to an example of the present disclosure.

FIG. 16 is a conceptual illustration of a bezel of a display that includes features that assist in location of a scroll wheel of a GUI according to an example of the present disclosure.

FIG. 17 illustrates a transition between two types of controls displayed on the GUI according to an example of the present disclosure.

FIG. 18 is a flow diagram illustrating a method for communicating with an implantable electrical stimulator using a programmer.

### DETAILED DESCRIPTION

The clinician may program numerous sets of stimulation parameters during the trial and error process for finding an efficacious stimulation program. Accordingly, during the process, the clinician may shift attention numerous times between the programming device that sets the stimulation parameters and the patient who provides feedback on the affect of the stimulation parameters. Shifting attention numerous times during the trial and error process may be an inefficient and inconvenient technique for determining an efficacious stimulation program. Accordingly, the process for finding an efficacious stimulation program may benefit from a programming device that allows the clinician to change stimulation parameters without focusing on the programming device, and instead allows the clinician to focus on the patient.

In general, the disclosure describes a programming device that allows the clinician to change stimulation parameters without focusing on the programming device, and instead allows the clinician to focus on the patient. For example, the programming device of the present disclosure may allow the clinician to adjust stimulation parameters of an implantable electrical stimulator while at the same time observing the patient and focusing on interpreting patient feedback. A scroll wheel, or other graphical gesture-based input medium, may allow the clinician to readily adjust parameters without focusing complete attention on the programming device. The ability to efficiently receive feedback from the patient coupled with the ability to concurrently test stimulation parameters may result in a more efficient process for finding an efficacious stimulation program.

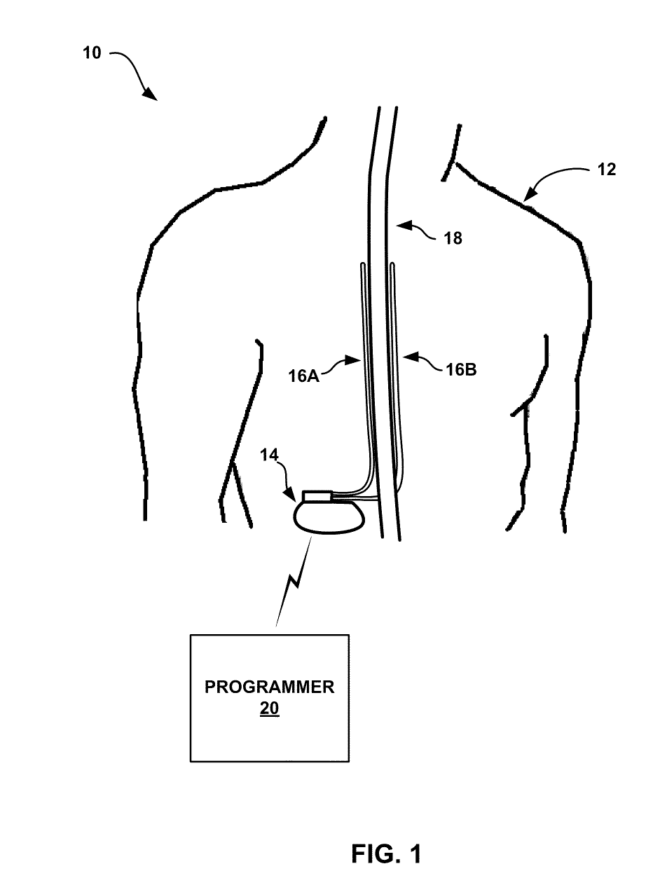

FIG. 1 is a conceptual diagram of an example system 10 for providing electrical stimulation therapy. In the example of FIG. 1, system 10 includes an implantable electrical stimulator 14 (hereinafter “stimulator 14”) and a medical device programmer 20 (hereinafter “programmer 20”). Stimulator 14 may be implanted within a patient 12 to deliver electrical stimulation therapy to patient 12. In other examples, stimulator 14 may be an external stimulator, e.g., an external neural stimulator, which may be used on a trial basis with percutaneous leads to test stimulation on patient 12. Programmer 20 programs stimulator 14. Although programmer 20 may be implemented as either a clinician programmer or a patient programmer, programmer 20 of the present disclosure will generally be described as a clinician programmer.

As shown in FIG. 1, stimulator 14 may be coupled to electrical leads 16A and 16B (collectively “leads 16”). Leads 16 include electrodes (not shown in FIG. 1) that deliver the electrical stimulation therapy to patient 12. Alternatively, in some implementations, stimulator 14 may be a leadless stimulator that includes electrodes on the housing of stimulator 14. In the example of FIG. 1, leads 16 are implanted along the length of spinal cord 18 such that electrical stimulation from leads 16 affects spinal cord 18. In other examples, one or more of leads 16 may be implanted to place the electrodes at target locations adjacent deep brain stimulation (DBS) targets, gastric nerves, pelvic nerves, peripheral nerves, and/or a variety of organs such as the heart, stomach, bladder, or the like. Although two leads 16 are shown in FIG. 1, in other implementations, system 10 may include more or less than two leads 16 implanted within patient 12. In some examples, leads 16 may be in the form of paddle leads or other shapes different than that shown in FIG. 1.

Leads 16 may include electrical and mechanical connectors at a proximate end of leads 16 that connect leads 16 to stimulator 14. Leads 16 include one or more electrodes along the length of leads 16 and/or proximate to distal ends of leads 16. As illustrations, the electrodes may be arranged as rings or segments in the case of cylindrical leads, or pads in the case of paddle leads.

Although programmer 20 and stimulator 14 are used in a spinal cord stimulation (SCS) system as shown in FIG. 1, other systems are contemplated. For example, as noted above, programmer 20 and stimulator 14 may be used in deep brain stimulation (DBS), gastric stimulation, pelvic nerve stimulation (e.g., sacral, pudendal, iliohypogastric, ilioinguinal, dorsal, peritoneal, or the like), peripheral nerve stimulation, peripheral nerve field stimulation (e.g., occipital, trigeminal, or the like), or any other type of electrical stimulation therapy. Although the configuration and/or location of stimulator 14 and/or leads 16 may be different depending on the specific application of system 10, programmer 20 may still function according to the description herein.

Stimulator 14 delivers electrical stimulation according to a set of stimulation parameters. Stimulation parameters may include voltage or current pulse amplitudes, pulse widths, pulse rates, electrode combination, and electrode polarity. Pulse amplitude may refer to the intensity or strength of a pulse, measured in volts or amperes. Pulse width may refer to a duration of a stimulation pulse, measured in microseconds (μs). Pulse rate may refer to a number of times per second that a stimulation pulse is delivered, measured in pulses per second or in Hertz (Hz). Electrode polarity refers to the ability of stimulator 14 to set each electrode as either an anode or a cathode. Additionally, electrode polarity may refer to the ability of stimulator 14 to set an electrode to an “off” state. Selection of electrode polarity and selection of whether an electrode is on/off allows for selection of multiple electrode configurations. A combination of the stimulation parameters listed above may be referred to as a “stimulation program.” Accordingly, a stimulation program may include settings for electrode configurations, pulse amplitude, pulse width, and pulse rate. A program may be stored in stimulator 14 and/or programmer 20. Multiple stimulation programs may be combined into a program group. Stimulator 14 may provide stimulation according to the program group. For example, stimulator 14 may deliver pulses according to a program group by sequentially delivering pulses from each of the programs of the program group, e.g., on a time-interleaved basis.

Using programmer 20, a user (e.g., a clinician) may create one or more customized programs that define the electrical stimulation delivered to patient 12 by stimulator 14. Programmer 20 may transmit the programs created by the clinician to stimulator 14. Stimulator 14 subsequently generates and delivers electrical stimulation therapy according to the programs created by the clinician to treat a variety of patient conditions such as chronic pain. In other examples, stimulator 14 may deliver electrical stimulation therapy to address a variety of symptoms or conditions such as tremor, Parkinson's disease, epilepsy, urinary or fecal incontinence, sexual dysfunction, obesity, or gastroparesis.

The clinician may directly adjust stimulation parameters. Alternatively, for some stimulation parameters, the clinician may interact with programmer 20 to create a visual representation of stimulation to be delivered by stimulator 14 to patient 12. For example, programmer 20 may present a visual representation of distributions of amplitude levels among electrodes in an electrode combination used to deliver stimulation. A group of one or more cathodes, for example, may be indicated by a cathodal control shape that represents a proportional distribution of current or voltage amplitude among the cathodes in the group. Similarly, an anodal control shape may be displayed to represent a proportional distribution of current or voltage amplitudes among a group of anodes. The user may manipulate the control shapes to adjust the distribution of amplitudes among the anodes or cathodes and, in some cases, add or subtract anodes or cathodes from the respective groups. Programmer 20 may then automatically generate stimulation parameters based on the created control shapes and transmit the stimulation parameters to stimulator 14, e.g., as a program. For example, the representation of the control shape may be mapped to or correlated with the stimulation parameters to produce the stimulation field in patient 12. In some cases, the clinician may have the capability to manipulate a control shape to indirectly adjust stimulator parameters (e.g., by implicit adjustment via manipulation of the control shape) as well as the capability to directly adjust stimulation parameters (e.g., by explicitly adjusting values), such as amplitude, pulse width, pulse rate, and/or electrode configuration.

Programmer 20 communicates with stimulator 14 via wireless communication. For example, programmer 20 may communicate with stimulator 14 during initial programming of stimulator 14, during follow-up programming, or to retrieve data collected by stimulator 14. For example, data collected by stimulator 14 may include a status of the battery, electrical operational status, lead impedance, and sensed physiological signals. Wireless communication between programmer 20 and stimulator 14 may include radio-frequency (RF) communication according to standard or proprietary RF telemetry protocols for medical devices, or other technique such as telemetry according to Institute of Electrical and Electronics Engineers (IEEE) 802.11, Bluetooth specification sets, or other standard or proprietary telemetry protocols.

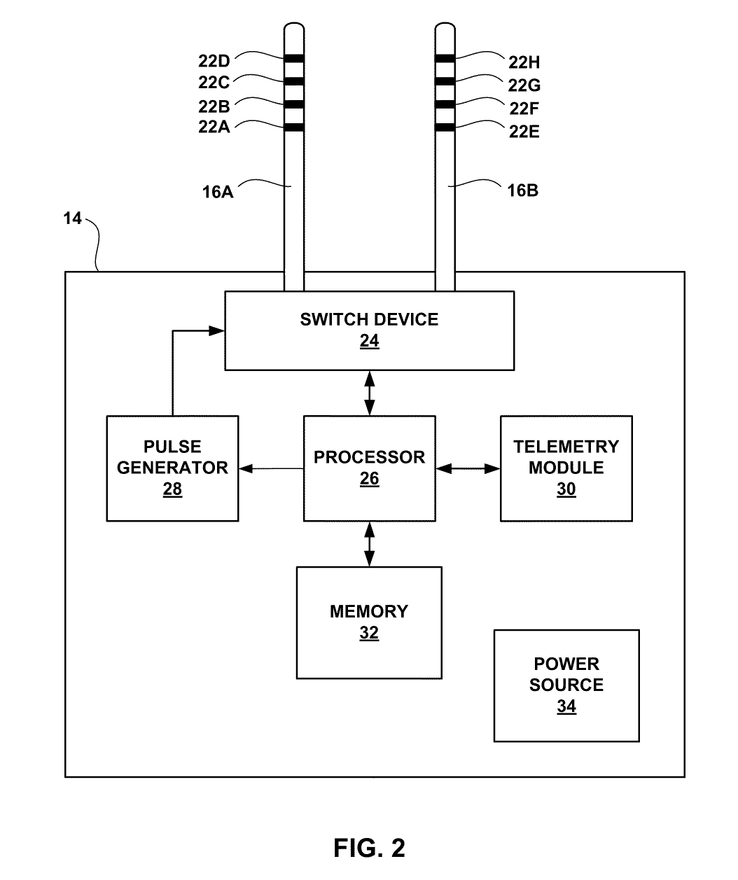

FIG. 2 is a functional block diagram of stimulator 14. Stimulator 14 may deliver stimulation via electrodes 22A-D of lead 16A and electrodes 22E-H of lead 16B (collectively “electrodes 22”). Electrodes 22 may be ring electrodes that form a cylinder around the exterior of leads 16. Alternatively, electrodes 22 may have other geometries such as pad electrodes arranged on a paddle lead. Electrodes 22 may also be segmented electrodes arranged in segments or sections around the circumference of leads 16. In some cases, ring electrodes, pad electrodes, partial ring electrodes, and/or segmented electrodes may be combined on a single lead. The configuration, type, and number of electrodes 22 and leads 16 illustrated in FIG. 2 are merely exemplary. Stimulator 14 may deliver stimulation via various other lead and electrode configurations. For example, a single lead may be used that includes 4, 8, or 16 electrodes. Alternatively, two leads may be used that include 4, 8, or 16 electrodes each. In some cases, three or more leads may be used, each having different electrode counts.

Electrodes 22 are electrically coupled to a switch device 24. A processor 26 controls switch device 24 to selectively couple each of electrodes 22 to a pulse generator 28. In some implementations, switch device 24 and pulse generator 28 may be replaced by separate pulse generators 28 that are each coupled to an electrode 22. Alternatively, in other implementations, stimulator 14 may include multiple pulse generators 28 that are coupled to electrodes 22 using one or more switch devices 24. In some examples, stimulator 14 may include electronic hardware that produces continuous waveforms, such as sine waves.

In some implementations, pulse generator 28 may be voltage based and each electrode may be coupled to its own regulated voltage source. In other implementations, pulse generator 28 may be current based and each electrode may be coupled to its own regulated current source. In still other implementations, hybrid arrangements of electrodes may share current sources on a multiplexed basis and share voltage sources on a multiplexed basis. Additionally, electrodes may be selectively coupled to a regulated source or selectively coupled to an unregulated source.

Pulse generator 28 may deliver electrical pulses to patient 12 via electrodes 22. Processor 26 controls pulse generator 28 to deliver the pulses according to stimulation parameters of a current program. Processor 26 controls switch device 24 to control which of electrodes 22 delivers pulses from pulse generator 28. Additionally, processor 26 controls switch device 24 to control the polarity of the pulses from pulse generator 28. The programs used by processor 26 to control pulse generator 28 and switch device 24 may be received via a telemetry module 30 and/or stored in memory 32. For example, the programs may be received from programmer 20.

Processor 26 may include a microprocessor, a microcontroller, a DSP, an ASIC, an FPGA, discrete logic circuitry, or the like, or any combination of one or more of the foregoing devices or circuitry. Memory 32 may include any volatile, non-volatile, or electrical media, such as RAM, ROM, NVRAM, EEPROM, flash memory, and the like. In some examples, memory 32 stores program instructions that, when executed by processor 26, cause stimulator 14 to perform the functions attributed to stimulator 14 herein.

Telemetry module 30 may include components to send data to and/or receive data from programmer 20. Telemetry module 30 may use any number of proprietary wireless communication protocols known in the medical device arts. Furthermore, telemetry module 30 may use RF signals according to any of a variety of standard or proprietary RF telemetry protocols for medical devices.

Power source 34 provides power to stimulator 14. Power source 34 may be a rechargeable or non-rechargeable battery, for example. Power source 34 may be recharged via inductive coupling, e.g., with programmer 20, when power source 34 is a rechargeable battery. In some implementations, power source 34 may use inductive coupling to an outside energy source to operate stimulator 14. In other words, in some implementations, power source 34 may not store adequate power for non-coupled operation of stimulator 14.

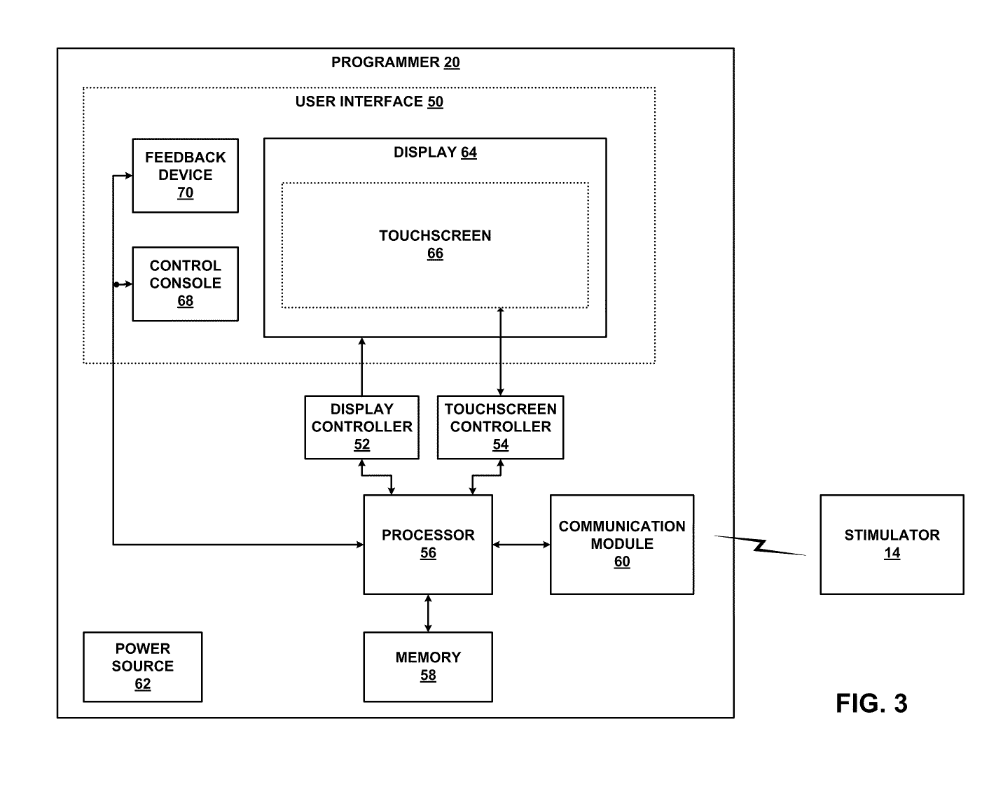

FIG. 3 is a functional block diagram of programmer 20. Programmer 20 includes a user interface 50, a display controller 52, a touchscreen controller 54, a processor 56, memory 58, a communication module 60, and a power source 62. Although display controller 52 and touchscreen controller 54 are illustrated in FIG. 3 as separate from processor 56, the functionality of display controller 52 and touchscreen controller 54 may be implemented by processor 56. Programmer 20 may be a dedicated hardware device with dedicated software for communicating with stimulator 14. For example, programmer 20 may be a dedicated hardware device that programs stimulation parameters of stimulator 14 and/or receives data from stimulator 14. Alternatively, programmer 20 may be an off-the-shelf computing device, such as a personal digital assistant (PDA), a desktop computer, a laptop computer, or a tablet-based computer running an application that enables programmer 20 to communicate with stimulator 14, i.e., program stimulator 14 and/or receive data from stimulator 14. Accordingly, programmer 20 may represent any computing device capable of performing the functions attributed to programmer 20 in the present disclosure. In some implementations, components of programmer 20 may be housed in a single housing such as, for example, a molded plastic housing. For example, user interface 50, display controller 52, touchscreen controller 54, processor 56, memory 58, communication module 60, and power source 62 may be housed in the housing. When housed in the single housing, in some examples, programmer 20 may be embodied as a hand-held computing device that the clinician may easily transport throughout the clinic, hospital, or any other location.

The clinician interacts with programmer 20 using user interface 50. User interface 50 includes a display 64 (e.g., a liquid crystal display (LCD)), a touchscreen 66, a control console 68, and a feedback device 70. The combination of display 64 and touchscreen 66 may be referred to as a “touchscreen display.” The clinician may enter data and/or commands into programmer 20 using control console 68 and touchscreen 66. Control console 68 may include various devices for controlling programmer 20 and entering data into programmer 20. For example, control console 68 may include a keypad such as, for example, an alphanumeric keypad or a reduced set of keys associated with particular functions of programmer 20. Control console 68 may also include a pointing device such as a mouse or a trackball.

Programmer 20 may provide feedback to the user via feedback device 70. For example, feedback device 70 may include, but is not limited to, a speaker to provide audible feedback and a vibrating device to provide tactile feedback, sometimes referred to as “haptic” feedback. Accordingly, the clinician may receive audible feedback, tactile feedback, or both from feedback device 70. In addition, in some examples, the clinician may receive visible feedback from display 64.

The clinician may enter data and/or commands into programmer 20 and control stimulator 14 using touchscreen 66, which may be overlaid or underlaid, relative to display 64, such that the user may interact with the display to enter user input such as data and/or commands. In general, display 64 may display a variety of information to the clinician and present a variety of controls for the clinician to interact with as described in this disclosure. For example, display 64 may display current stimulation parameters being applied by stimulator 14, such as voltage or current pulse amplitudes, pulse widths, pulse rates, and electrode configurations. Display 64 may also show a visual representation of leads, electrodes, and corresponding control shapes associated with the leads and electrodes. In some cases, programmer 20 may be configured to cause display 64 to present a graphical representation of a stimulation field produced by the stimulation delivered by stimulator 14.

Display 64 may also show graphical icons that the clinician may use (i.e., touch) to control programming of stimulator 14. Graphical icons that the clinician may use to control programming of stimulator 14 may be referred to as “controls.” Accordingly, the clinician may adjust stimulation parameters being applied by stimulator 14 by using controls displayed on display 64. For example, controls may include, but are not limited to, a scroll wheel, a rotary control wheel, and an omni-directional touch pad as described herein. Some of the controls presented by display 64, such as a scroll wheel or control wheel, may operate as graphical, gesture-based input media that permit a clinician to adjust stimulation parameters by gesture-based input, such as swiping, tracing of a shape, or the like.

Display controller 52 displays graphical information on display 64. Display controller 52 receives graphical information from processor 56 and generates graphical images on display 64 based on the graphical information received from processor 56. For example, display controller 52 may generate images of stimulation parameters received from processor 56, controls (e.g., a scroll wheel), representations of leads and electrodes, and representations of patient 12.

Touchscreen 66 in conjunction with touchscreen controller 54 represents one or more touchscreen technologies, to be described hereinafter, that may determine where an object contacts a screen of display 64. Typically, touchscreen 66 includes a component that overlays the screen of display 64 and touchscreen controller 54 may be an electronic component that provides for detection of objects that touch touchscreen 66.

Touchscreen controller 54 may detect various types of interactions with the clinician. For example, touchscreen controller 54 may detect discrete interactions with touchscreen 66 and gesture based contact with touchscreen 66. Discrete interactions may include discrete selections made by the clinician, for example, using touchscreen 66 as a push button. In other words, the clinician may make a selection on display 64 by tapping on touchscreen 66, much in the same way as pushing a physical button. Accordingly, touchscreen 66 may be used as a keypad such as, for example, an alphanumeric keypad, similar to that described in respect to control console 68.

Touchscreen controller 54 may also detect gestures (i.e., gesture-based contact) made on display 64. For example, touchscreen controller 54 may, by tracking a touch on touchscreen 66 over a period of time, detect gestures made by an object on display 64. In one example, touchscreen controller 54 may detect when the clinician makes a swiping gesture on display 64. A swiping gesture may include touching display 64 (e.g., using a finger) at a first point, then moving a finger from the first point to a second point while maintaining contact with display 64. Touchscreen controller 54 may determine the speed and direction of a swiping gesture. Touchscreen controller 54 may determine the speed of the swiping gesture based on a total distance between the first and second points divided by a total time in which display 64 was contacted during the swiping gesture. The direction of the swiping gesture may be determined based on coordinates of the first and second points on display 64. Processor 56 may communicate with touchscreen controller 54 to detect the various types of interactions (e.g., discrete or gesture based) between the clinician and touchscreen 66.

Touchscreen 66 may include various touchscreen technologies. Although touchscreen 66 may be implemented using a technology that is responsive to physical touching, e.g., with the user's finger and/or stylus, other technologies that do not require contact with a user's finger or stylus are contemplated, such as the pen digitizing technology described herein.

Touchscreen 66 may include, but is not limited to, one or more of the following touchscreen technologies: a resistive technology, a capacitive technology, and a pen digitizing technology. Each of these example touchscreen technologies and implementation of the touchscreen technologies in programmer 20 are now discussed in turn.

The resistive touchscreen technology, for example, may include a touchscreen having flexible sheets separated by an air gap. The flexible sheets may be coated with conductive material that forms contacts between the sheets when the sheets are pressed together. Touchscreen controller 54 may detect where the flexible sheets contact each other and accordingly, may determine where touchscreen 66 is touched. The flexible sheets of a resistive touchscreen may be transparent and therefore may be laid over display 64 without interfering with images on display 64 as viewed by the clinician. The resistive touchscreen may be actuated by pressure, and accordingly, an insulating or a conductive object may activate touchscreen 66 that includes resistive touchscreen technology. Accordingly, the clinician may operate touchscreen 66 with or without insulative gloves (e.g., latex gloves). The clinician may also operate the touchscreen using an object, such as a stylus.

A capacitive touchscreen technology may include, for example, a conductor coated over an insulator, such as the glass screen covering display 64. For example, the glass screen covering display 64 may be patterned with a conductive material to form a capacitive touchscreen. Touchscreen controller 54 may detect contact (e.g., with the clinician's finger) with the capacitive touchscreen based on a change in measured capacitance during a contact between an object and the touchscreen. The conductor coated glass may be transparent and therefore may be laid over display 64 without interfering with graphical images on display 64 as viewed by the clinician. In some implementations, the capacitive touchscreen technology may not operate if the clinician's hand is covered, for example, while wearing insulative gloves.

Touchscreen 66 and touchscreen controller 54 may comprise a pen digitizing technology. An example pen digitizing technology may include a sensor board positioned behind display 64 that interacts with a pen-input device. In general, the sensor board may detect the position of the pen-input device based on a signal received from the pen-input device. Accordingly, the pen digitizing technology may be limited to detecting the position of the pen-input device, and may not detect contact between an object, such as a finger, and display 64.

The various touchscreen technologies described above, as well as other touchscreen technologies not described herein, may allow for detection of discrete interactions and gesture based interactions with touchscreen 66.

Some of the above touchscreen technologies may indicate pressure exerted on display 64 by the clinician. Accordingly, in some implementations, touchscreen controller 54 may determine an amount of pressure exerted on touchscreen 66 by the clinician. Therefore, the clinician may vary an amount of pressure applied to touchscreen 66 as a means to interact with programmer 20. For example, the clinician may apply a greater amount of pressure to effect a larger change in a stimulation parameter.

Processor 56 can take the form of one or more microprocessors, microcontrollers, DSPs, ASICs, FPGAs, programmable logic circuitry, or the like, and the functions attributed to the processor 56 herein may be embodied as hardware, firmware, software or any combination thereof. Processor 56 of programmer 20 may provide any of the functionality ascribed herein to programmer 20, or otherwise perform any of the methods described herein.

Processor 56 may control stimulator 14 via communication module 60 to test created stimulation programs. Specifically, processor 56 may transmit programming signals, based on communication with touchscreen controller 54, to stimulator 14 via communication module 60. Processor 56 may send one or more programs to stimulator 14 and stimulator 14 may deliver therapy according to the one or more programs without further input from programmer 20. Accordingly, processor 56 may communicate with stimulator 14 in real-time via communication module 60 so that the clinician may immediately observe the programming change in patient 12. In some cases, changes to stimulation parameters may not be immediately evident. In such cases, a change may be activated and evaluated over a period of minutes, hours, or days before another change is initiated.

Finalized programs may be transmitted by processor 56 via communication module 60 to stimulator 14. Alternatively, programs may be stored in stimulator 14 and modified or selected using instructions transmitted by processor 56 via communication module 60.

Memory 58 may store programs, including those created by the clinician or other user, e.g., patient 12, using the techniques described herein. Processor 56 may download the programs to stimulator 14 via communication module 60. Memory 58 may also store instructions that cause processor 56 to provide the functionality ascribed to programmer 20 herein.

Memory 58 may include any fixed or removable magnetic, optical, or electrical media, such as RAM, ROM, CD-ROM, hard or floppy magnetic disks, EEPROM, or the like. Memory 58 may also include a removable memory portion that may be used to provide memory updates or increases in memory capacities. A removable memory may also allow patient data to be easily transferred to another computing device, or to be removed before programmer 20 is used to program therapy for another patient. In some implementations, programmer 20 may include a device interface that provides for transfer of data from programmer 20 to another device for storage. For example, programmer 20 may store data on a networked storage device through a network interface, or to a local storage device using a universal serial bus (USB) interface.

Programmer 20 may communicate wirelessly with stimulator 14 using RF communication or proximal inductive interaction, for example. This wireless communication is possible through the use of communication module 60, which may be coupled to an internal antenna or an external antenna (not shown). Communication module 60 may include functionality similar to telemetry module 30 of stimulator 14.

Examples of local wireless communication techniques that may be employed to facilitate communication between programmer 20 and another computing device using communication module 60 may include RF communication according to the 802.11 or Bluetooth specification sets, infrared communication, e.g., according to the IrDA standard, or other standard or proprietary telemetry protocols.

Power source 62 delivers operating power to the components of programmer 20. Power source 62 may include a battery and/or adapter for connection to an alternating current (AC) wall socket.

In summary, display 64 displays graphical information to the clinician related to programming stimulation parameters of stimulator 14. Using touchscreen 66, the clinician may access various functions of programmer 20 to change stimulation parameters of programmer 20, which in turn change the stimulation parameters applied by stimulator 14 in real-time. In other words, the clinician may modify stimulation parameters using touchscreen 66, which may result in immediate modification of the stimulation parameters implemented by stimulator 14. Accordingly, in some examples, the clinician may modify stimulation parameters of stimulator 14 in real-time using touchscreen 66. Also, in some examples, programmer 20 may immediately transmit the modified parameters to stimulator 14 for delivery of modification stimulation therapy to the patient. In this case, the clinician may receive feedback from patient 12 regarding the affect of the change in the stimulation parameters on patient 12 substantially concurrently with such changes being made by the clinician via programmer 20. For example, the clinician may manipulate the amplitude of a voltage waveform being applied by stimulator 14 using touchscreen 66, and patient 12 may give a verbal response as to the affect of the manipulation of the amplitude. In other examples, the clinician may adjust the parameters and then enter additional input to cause programmer 20 to selectively transmit the resulting parameters to stimulator 14.

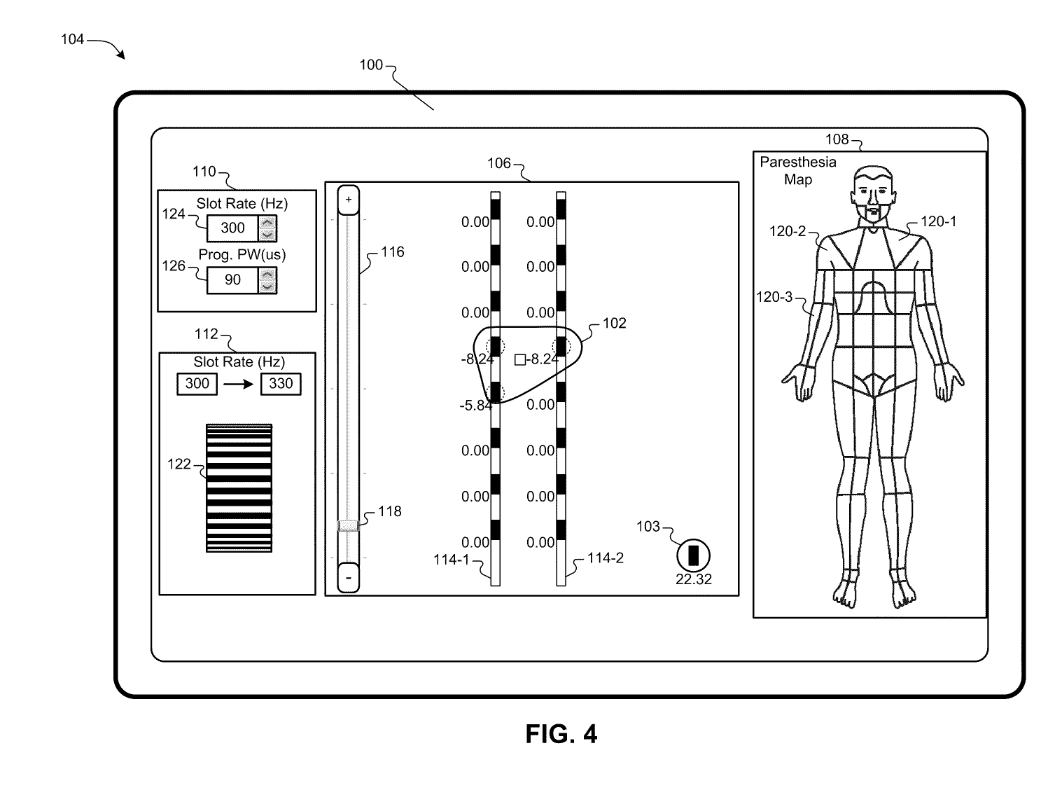

Techniques for interacting with stimulator 14 using user interface 50 will now be discussed in conjunction with example graphical user interfaces (GUIs) of FIGS. 4-16 that may be displayed on display 64.

FIGS. 4-16 are conceptual illustrations of GUIs displayed on display 64. The GUIs illustrated in FIGS. 4-16 facilitate programming of electrical stimulation therapy applied by stimulator 14 implanted in patient 12. In other implementations, the GUIs illustrated in FIGS. 4-16 may facilitate programming of other medical devices, such as stimulators that apply external electrical stimulation therapy. Display 64 of programmer 20 illustrated in FIGS. 4-10 and FIGS. 13-16 is surrounded by a bezel 100, e.g., a plastic bezel that surrounds the screen of display 64 and houses the components of programmer 20.

Programmer 20 may display various windows that convey information to the clinician regarding programming of stimulator 14. For example, in FIG. 4, programmer 20 displays information regarding pulse rate, pulse width, control shapes, or other parameters. Programmer 20 may also display controls on display 64 which the clinician can interact with using touchscreen 66. For example, the clinician may use discrete actions (e.g., a tap on the screen) or gesture-based actions (e.g., a swipe) as input in areas where controls are present on display 64 in order to control stimulator 14. Additionally, some controls may control various options on the user interface, e.g., zoom functions, annotation functions, etc, that do not evoke a change in stimulation parameters of stimulator 14.

Display 64 may display a control shape. A control shape may be an icon that is used by the clinician to specify proportional current or amplitude level contributions from electrodes associated with the control shape. Display 64 may present multiple control shapes. Each control shape may be a cathodal control shape, containing one or more cathodes, or an anodal control shape, containing one or more anodes.

In a bipolar or multipolar configuration, the leads may be displayed in conjunction with at least one cathodal control shape and at least one anodal control shape. In a unipolar configuration, a cathodal control shape may be presented in conjunction with a control shape presented in relation to a housing associated with stimulator 14. The housing may form, or carry, one or more anodes that form a so-called case or can anode. Alternatively, a unipolar arrangement could include one or more anodes on one or more leads and one or more can cathodes. In some examples, display 64 also may display a field representation simultaneously with the control shapes, or selectively as an alternative to presentation of control shapes. For example, in one implementation, the control shape may be representative of a current density that illustrates how the electrical current from the electrical field produced by electrodes 22 propagates or is expected to propagate through the tissue of patient 12 around leads 16. The control shape, or the resulting stimulation field shape, may be adjusted to illustrate any aspect of the stimulation therapy that would provide insight to the clinician for programming the stimulation therapy. Although gesture-based control is described in conjunction with the control shape methodology presented in FIGS. 4-16, other methodologies may be used to control stimulation parameters.

Programmer 20 may receive input from the clinician that manipulates the shape and/or position of the control shape. In response to such manipulation of shape and/or position, programmer 20 may automatically adjust stimulation amplitude contributions of the electrodes that deliver stimulation. Using various input media (e.g., a stylus or a finger), the clinician may size (e.g., by stretching or contracting), shape, or move the control shape. The user may shape, move, stretch, shrink, and expand the control shape by dragging, for example, the control shape to other areas, or zones. In one example, a zone may be stretched by clicking with a mouse or touching with a stylus, for example, within the control shape and then dragging the boundaries of the control shape. Changes produced by stretching may include changes in contribution and/or changes in the number of electrodes recruited by the control shape. As another example, a control shape may be stretched or shrunk by moving two fingers (e.g, thumb and forefinger) apart or together, respectively.

FIG. 4 shows an example GUI 104 that includes windows that display information related to programming stimulator 14 and documenting the response of patient 12. GUI 104 includes a lead display window 106, a paresthesia map 108, and a stimulation parameter window 110. GUI 104 also includes a control icon window 112. Information displayed in each of windows 106, 108, 110, and 112 will now be discussed in turn.

Lead display window 106 includes a representation of two implantable leads 114-1 and 114-2 implanted in a stimulation region of patient 12. Leads 114-1 and 114-2 include electrodes represented by the darkened regions of leads 114-1 and 114-2. The representation of leads 114-1 and 114-2 in lead display window 106 may be representative of leads 16 described in FIGS. 1 and 2.

In the example of FIGS. 4-10 and FIGS. 13-16, lead display window 106 includes a sliding control 116. Sliding control 116 may be used to zoom in and out on the representation of leads 114-1 and 114-2. For example, sliding control 116 may be adjusted to zoom in and out, and therefore change a number of electrodes viewed in lead display window 106. Slider 118 of sliding control 116 as shown in FIG. 4 is at the bottom of sliding control 116 near the (−) symbol. Accordingly, the view of leads 114-1 and 114-2 may be zoomed out to show the entire set of eight electrodes on each of the leads 114-1 and 114-2.

Lead display window 106 includes a control shape 102. Control shape 102 is positioned around electrodes of leads 114-1 and 114-2. Control shape 102 includes three active electrodes as illustrated by the dotted circles. The numbers next to the active electrodes (i.e., −8.24, −8.24, and −5.84) may represent an amplitude associated with the stimulation field. In the example of FIGS. 4-16, the control shape 102 illustrates a cathodal control shape comprising three cathodes. In this example, the cathodal control shape 102 represents a unipolar configuration, in conjunction with an anode provided by a housing associated with stimulator 14.

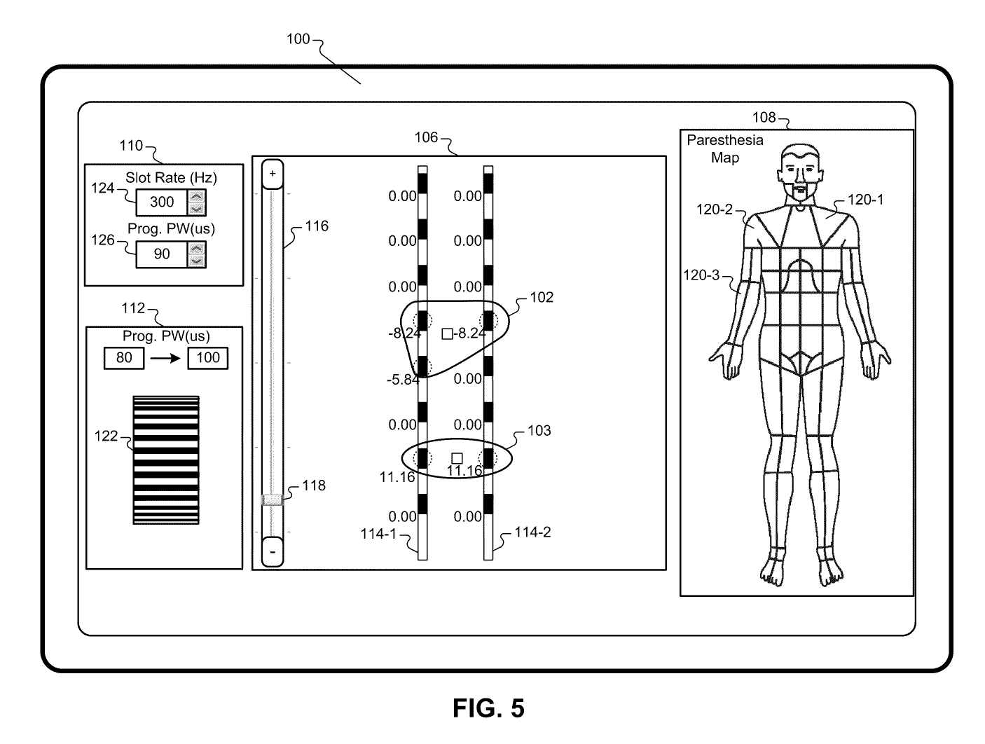

An anodal control shape 103 provided by the housing is illustrated in FIGS. 4, 6-10, and 13-16. Alternatively, or additionally, the anodal control shape 103 may be implemented using the electrodes on leads 114-1 and 114-2 (i.e., in a bipolar configuration), as shown in FIG. 5. For example, in FIG. 5, anodal control shape 103 is illustrated as comprising two anodes. Although a single anodal control shape 103 and cathodal control shape 102 are illustrated, more anodal and cathodal control shapes may be added to the leads 114-1 and 114-2. For example, anodal control shapes may be added above and below the cathodal control shape 102. Although the cathodal control shape 102 is illustrated as including three electrodes and the anodal control shape 103 is illustrated as including one or two electrodes, the cathodal control shape 102 and the anodal control shape 103 may be adjusted to include any number of electrodes.

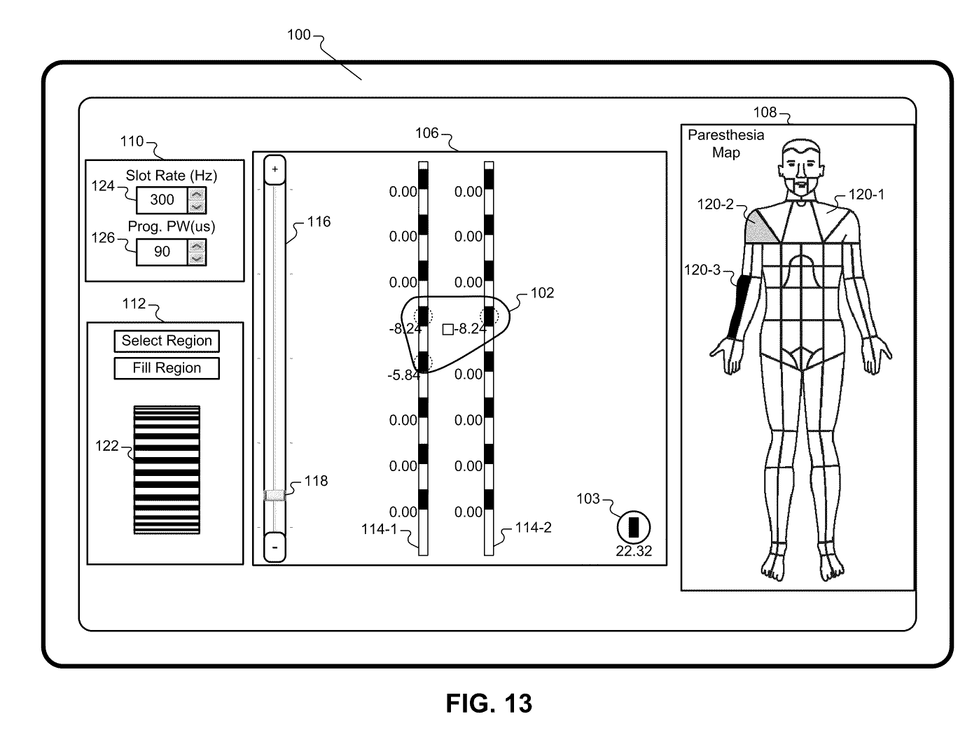

Paresthesia map 108, in the example of FIGS. 4-10 and 13-16, displays a mapping of patient 12 that includes sections 120-1, 120-2, and 120-3. Although paresthesia map 108 illustrates sections 120-1, 120-2, and 120-3 on a front of patient 12, a radio selection button (i.e., the radio button labeled “posterior view”) may be selected to show a posterior view of patient 12 that includes sections on the posterior of patient 12. As described herein, each of the sections on the paresthesia map may be colored by the clinician, for example, to indicate an amount of paresthesia and/or pain felt by patient 12. Accordingly, using touchscreen 66, the clinician may mark the sections of paresthesia map 108 according to verbal feedback from patient 12 in real-time as the stimulation parameters are manipulated. Navigation of paresthesia map 108 and coloring of the sections of paresthesia map 108 to indicate a location and amount of paresthesia/pain felt by patient 12 is further described in conjunction with FIG. 13.

Stimulation parameter window 110 may display current stimulation parameter values being used by stimulator 14. For example, stimulation parameter window 110 of FIG. 4 illustrates that the current slot rate is set at 300 Hz and the current programmed pulse width is set at 90 μs for a program assigned to the slot. Stimulation parameter window 110 may update the slot rate and the programmed pulse width in real-time as new values are modified using the control of control icon window 112. Slot rate may be a parameter that is defined when using a slot-based programming technique. Specifically, slot rate may be the rate at which the pulses for a program assigned to a slot are delivered. In slot-based programming, instead of forming program groups, n therapy slots are defined, where each therapy slot may be occupied by one of m programs. Each therapy slot may be associated with therapy directed to a particular condition and/or anatomical region (e.g., left leg pain, lower back pain, etc.).

Control icon window 112 includes a control 122. Control 122 illustrated in FIG. 4 represents a scroll wheel. Control 122 may also include, but is not limited to, a rotary control wheel, and an omni-directional touch pad as described in this disclosure. Above control 122 are a range of values associated with a particular stimulation parameter. The range of values may be adjusted using control 122. For example, control 122 of FIG. 4 may be configured to adjust the slot rate from 300-330 Hz, depending on how the clinician interacts with control 122. In response to interaction with control 122, processor 56 may adjust the slot rate to a value between 300-330 Hz.

The clinician may select other stimulation parameters that may be controlled using control 122. For example, the clinician may select pulse width, and subsequently adjust pulse width using control 122, as illustrated in FIG. 5. In other implementations, the clinician may also control the amplitude of the pulses. In still other implementations, the clinician may control the location of the pulses by changing the electrode configuration used by stimulator 14 using control 122.

The clinician may select the stimulation parameter to adjust by touching touchscreen 66 in a specific area. For example, the clinician may select the slot rate parameter by touching the current slot rate indicator 124. The clinician may select the pulse width parameter by touching the current pulse width indicator 126. The clinician may select the amplitude, for example, by touching control shape 102. Current slot rate indicator 124, current pulse width indicator 126, and control shape 102 may be highlighted when selected to indicate to the clinician which parameter is being adjusted by control 122.

Control 122 shown in FIG. 4 represents a scroll wheel. Accordingly, control 122 may be referred to as a “scroll wheel 122.” The clinician may control scroll wheel 122 using touchscreen 66. For example, the clinician may touch touchscreen 66, e.g., using their finger, over scroll wheel 122 and drag their finger either up or down scroll wheel 122 to spin scroll wheel 122. Scroll wheel 122 is oriented vertically, and accordingly, the clinician may actuate (i.e., rotate) scroll wheel 122 by making a vertical swiping motion over scroll wheel 122. Although scroll wheel 122 is described herein as being actuated with a clinician's finger, the clinician may use other objects in addition to their finger to actuate scroll wheel 122 or any other control in the GUI. For example, the clinician may use a stylus to actuate scroll wheel 122.

The clinician may actuate scroll wheel 122 in order to adjust stimulation parameters of stimulator 14. Specifically, as shown in FIG. 4, the user may actuate scroll wheel 122 to adjust the slot rate. The slot rate may be adjusted within the limits (i.e., 300 Hz and 330 Hz) listed above scroll wheel 122. The values listed above scroll wheel 122 indicate maximum and minimum threshold values (collectively “threshold values”) for the stimulation parameter (i.e., prog. PW) listed above scroll wheel 122. Accordingly, the threshold values may be maximum and minimum values to which scroll wheel 122 may adjust the listed stimulation parameter. For example, in FIG. 4, scroll wheel 122 may be used to adjust the slot rate from 300 Hz to 330 Hz, and in FIG. 5, scroll wheel 122 may be used to adjust the pulse width from 80-100 μs.

The maximum and minimum thresholds may be set by the clinician. For example, the clinician may enter the maximum and minimum thresholds using control console 68, i.e., a numeric keypad. Alternatively, processor 56 may determine the maximum and minimum thresholds based on current values of other stimulation parameters.

In some examples, a rate of increase of a stimulation parameter may be set by the user. For example, a rate of increase of amplitude may be limited to 1 Volt or 1 mA per second. Similarly, an increase in pulse width and/or pulse rate may be subject to a rate limitation. Stimulation parameters that are subject to a rate limitation when increased may not be subject to a rate limitation when decreased. In other words, a decrease in amplitude, pulse width, or pulse rate may be realized immediately in response to input from the user. Although a rate of increase may be set by the user in some examples, as described above, in other examples, a rate limit may not be set for an increase or a decrease. In other words, stimulation parameters may not be subject to a rate limitation when a parameter is increased or decreased.

In some examples, the rate limit set for an increase in a stimulation parameter may be dependent on the current magnitude of the parameter relative to the maximum threshold corresponding to the parameter. In other words, the rate limit may differ based on how close the current magnitude of the stimulation parameter is to the maximum threshold. For example, if the current amplitude is set at 1 mA and the maximum threshold is set to 4 mA, amplitude may be adjusted by 2-3 mA per second until the amplitude reaches 3 mA, then subsequently, the rate of increase of the amplitude may be set at 0.1 mA per second until the amplitude reaches 4 mA. Accordingly, a rate limit that is dependent on the current magnitude relative to the maximum threshold may allow for a quicker and more coarse adjustment when the magnitude is further from the maximum threshold, and allow for a finer tuning of the magnitude when the magnitude is closer to the maximum threshold.

Scroll wheel 122 may be configured to operate based on various scroll wheel parameters. The sensitivity of scroll wheel 122 may be adjusted. Sensitivity of scroll wheel 122 may refer to an amount of change in the stimulation parameter in response to actuation of scroll wheel 122. When sensitivity of scroll wheel 122 is increased, a greater change in the controlled stimulation parameter per unit of movement of scroll wheel 122 may result. When sensitivity of scroll wheel 122 is decreased, a lesser change in the controlled stimulation parameter per unit of movement of scroll wheel 122 may result.

Sensitivity of scroll wheel 122 may also be set in terms of a stepping value associated with the stimulation parameter. In other words, the changes in the selected stimulation parameter may be made in discrete steps in response to actuation of scroll wheel 122. For example only, the slot rate may be set in steps of 5 Hz. Accordingly, if the slot rate of FIG. 4 was set to step in 5 Hz intervals, the slot rate would be adjustable from 300 Hz to 330 Hz in stepping increments of 5 Hz in response to actuation of scroll wheel 122.

The darkened horizontal bars of scroll wheel 122 may move in the direction of actuation to give the appearance that scroll wheel 122 is rotating. The number of horizontal bars that move out of the user's field of view may correspond to a number of discrete steps made in the selected stimulation parameter. In some implementations, the selected parameter may be incremented/decremented by one step for each horizontal bar that passes out of the user's field of view. For example, scroll wheel 122 may increase/decrease the selected parameter by one step per horizontal bar that passes out of the user's field of view. In other implementations, the selected parameter may be incremented/decremented by one step only after a plurality of horizontal bars has passed out of the user's field of view. For example, scroll wheel 122 may increase/decrease the selected parameter by one step per every three horizontal bars. Accordingly, in some implementations, scroll wheel 122 may increase/decrease the selected parameter by 10 steps per revolution of scroll wheel 122 when scroll wheel 122 includes 30 horizontal bars per revolution.

Scroll wheel 122 may include an inertia parameter that causes scroll wheel 122 to continue to rotate after scroll wheel 122 is actuated. For example, the clinician may make a swiping motion (i.e., a swipe) across scroll wheel 122 and scroll wheel 122 may continue to rotate after the swipe. The amount of rotation after the swipe may depend on the amount of inertia associated with scroll wheel 122 and the speed of the swipe. When scroll wheel 122 has a greater amount of inertia, scroll wheel 122 may rotate for a shorter period of time after being swiped from a resting position, while a scroll wheel having a lesser amount of inertia may rotate for a greater period of time after being swiped from a resting position.

A speed of the swipe that actuates scroll wheel 122 may affect the amount of rotation of scroll wheel 122 after the swipe. A scroll wheel that has been swiped at a greater speed may continue to rotate for a longer period after being swiped, while a scroll wheel that has been swiped at a lesser speed may continue rotating for a relatively shorter period after being swiped. Accordingly, adjustment of stimulation parameters after swiping scroll wheel 122 may be based on the speed of the swipe that actuates scroll wheel 122 and an amount of inertia associated with scroll wheel 122.

Based on the above description of the affect of swiping speed and inertia on the behavior of scroll wheel 122, a few scenarios describe how swiping speed and inertia of scroll wheel 122 may affect stimulation parameters after swiping of scroll wheel 122. In general, a greater swiping speed may result in a greater change in stimulation parameters after swiping of scroll wheel 122. In general, a lesser resting inertia associated with scroll wheel 122 may result in a greater change in stimulation parameters after swiping of scroll wheel 122 when scroll wheel 122 is at rest.

In some implementations, the clinician may stop scroll wheel 122 from spinning after swiping scroll wheel 122. For example, the clinician may tap on scroll wheel 122 while scroll wheel 122 is spinning to stop scroll wheel 122 from spinning. In other implementations, the clinician may press and hold on scroll wheel 122 to stop scroll wheel 122 from spinning. In still other implementations, the clinician may tap anywhere on the screen in order to stop scroll wheel 122 from spinning after a swipe. Tapping anywhere to stop scroll wheel 122 is an action that may be easily performed by the clinician without looking directly at the screen. Accordingly, the clinician may focus on patient 12 while controlling stimulation parameters (i.e., while stopping scroll wheel 122) when tapping of the screen stops scroll wheel 122.

Although scroll wheel 122 may include an inertia parameter, in some implementations, scroll wheel 122 may not include an inertia parameter and therefore may not continue spinning after a swipe by the clinician. Accordingly, in some implementations, scroll wheel 122 may stop spinning, and therefore stop adjusting stimulation parameters, after the clinician removes their finger from touchscreen 66.

Programmer 20 may provide feedback to the clinician while the clinician operates scroll wheel 122. Both display 64 and feedback device 70 may provide feedback to the clinician. Display 64 may provide visual feedback during actuation of scroll wheel 122. For example, scroll wheel 122 may be animated to represent a rotating scroll wheel when scroll wheel 122 is actuated. When animated, the darkened horizontal bars of scroll wheel 122 may move in the direction of actuation to give the appearance that scroll wheel 122 is rotating. In addition to the animation of scroll wheel 122, the numbers presented on display 64 may provide feedback to the clinician. The numbers on display 64 may be updated as the stimulation parameters are adjusted by scroll wheel 122. For example, as shown in FIG. 4, the number “300” in current slot rate indicator 124 may be updated as scroll wheel 122 is actuated.

Feedback device 70 may include, but is not limited to, a speaker and a vibrating device. Accordingly, feedback device 70 may provide audible and/or tactile feedback. In general, audible feedback may include sounds such as beeping, clicking of the scroll wheel, etc. Tactile feedback may include vibration, e.g., a vibrating device in programmer 20 may vibrate so that the clinician holding programmer 20 may sense the vibration.

Audible feedback may include sounds that indicate whether the clinician's finger is touching scroll wheel 122. For example, feedback device 70 may provide a noise (e.g, a beep) that indicates when the clinician is contacting scroll wheel 122. Such audible feedback may allow the clinician to visually observe patient 12 without requiring the clinician to look back at display 64 to determine whether their finger is located on scroll wheel 122. In other words, based on the audible feedback produced when touching scroll wheel 122, the clinician may be assured that their finger is placed over scroll wheel 122 without looking at display 64.

Alternatively, or additionally, tactile feedback may provide a vibration that indicates when the clinician is contacting scroll wheel 122. Such tactile feedback may allow the clinician to visually observe patient 12 without requiring the clinician to look back at display 64 to determine whether their finger is located on scroll wheel 122. In other words, based on the tactile feedback (e.g., vibration) produced when touching scroll wheel 122, the clinician may be assured that their finger is placed over scroll wheel 122 without looking at display 64.

Audible feedback may indicate to what extent (i.e., a speed) scroll wheel 122 is being actuated. In other words, audible feedback may indicate a rate at which the stimulation parameters are being changed by scroll wheel 122. For example, feedback device 70 may provide a clicking noise that indicates how fast the clinician is rotating scroll wheel 122. Feedback device 70 may produce a clicking noise at a greater rate (i.e., number of clicks per second) to indicate a greater speed of rotation of scroll wheel 122. Feedback device 70 may decrease the rate of the clicking noise to indicate a reduced speed of rotation of scroll wheel 122. In some implementations, feedback device 70 may produce a clicking noise for each hash mark on scroll wheel 122 as the hash mark moves out of a field of view. Such audible feedback indicating a speed of rotation of scroll wheel 122 may allow the clinician to visually observe patient 12 without requiring the clinician to look back at display 64 to determine the rate at which scroll wheel 122 is being rotated. In other words, based on the audible feedback that indicates a speed of rotation of scroll wheel 122, the clinician may determine at what rate the stimulation parameters are being adjusted without looking at display 64.

Alternatively, or additionally, tactile feedback (e.g., vibrational feedback) may indicate to what extent (i.e., a speed) scroll wheel 122 is being actuated. In other words, tactile feedback may indicate a rate at which the stimulation parameters are being changed by scroll wheel 122. Feedback device 70 may provide a vibration that indicates how fast the clinician is rotating scroll wheel 122. For example, a single discrete vibration may correspond to a predetermined amount of rotational movement of scroll wheel 122, while a series of vibrations during a period of time may indicate how fast scroll wheel 122 is being rotated. In other words, feedback device 70 may produce vibrations at a greater rate (i.e., number of discrete vibrations per second) to indicate a greater speed of rotation of scroll wheel 122. Feedback device 70 may decrease the rate of the vibrations to indicate a reduced speed of rotation of scroll wheel 122. Such tactile feedback indicating a speed of rotation of scroll wheel 122 may allow the clinician to visually observe patient 12 without requiring the clinician to look back at display 64 to determine at what rate scroll wheel 122 is being rotated. In other words, based on the tactile feedback that indicates a speed of rotation of scroll wheel 122, the clinician may determine at what rate the stimulation parameters are being adjusted without looking at display 64.

Audible feedback may also indicate in which direction scroll wheel 122 is being rotated. In other words, audible feedback may indicate whether the stimulation parameter being adjusted by scroll wheel 122 is increasing or decreasing in value. For example, different clicking noises (e.g., a frequency content of sound associated with each click) may be provided that indicate rotational direction of scroll wheel 122, and in turn indicate whether the stimulation parameters are being increased or decreased. For example, a lower frequency click may indicate a decrease in stimulation parameter values, while a higher frequency click may indicate an increase in stimulation parameter values. Such audible feedback indicating in which direction scroll wheel 122 is being rotated may allow the clinician to visually observe patient 12 without requiring the clinician to look back at display 64 to determine which direction scroll wheel 122 is being rotated. In other words, based on the audible feedback that indicates in which direction scroll wheel 122 is being rotated, the clinician may determine whether the stimulation parameters are being increased or decreased without looking at display 64.

Audible feedback may indicate when scroll wheel 122 is being actuated to provide an adjustment that is prohibited by the minimum or maximum thresholds. In other words, audible feedback may indicate when the stimulation parameter being adjusted has reached the maximum/minimum threshold corresponding to the stimulation parameter. For example, feedback device 70 may produce a beeping noise to indicate when the maximum/minimum threshold has been reached. Such audible feedback indicating when the adjustment of scroll wheel 122 is prohibited by the maximum/minimum thresholds may allow the clinician to visually observe patient 12 without requiring the clinician to look back at display 64 to determine whether the maximum/minimum thresholds have been achieved. In other words, based on the audible feedback that indicates when the maximum/minimum thresholds have been reached, the clinician may determine when the maximum/minimum values for the stimulation parameters have been reached without looking at display 64.

In some implementations, feedback device 70 may provide tactile feedback to indicate when the stimulation parameter being adjusted has reached the maximum/minimum threshold. For example, feedback device 70 may not provide tactile feedback to indicate any of the above mentioned operations (e.g., contact/speed/direction of scroll wheel 122) but may provide feedback when the stimulation parameter being adjusted has reached the maximum/minimum threshold. In other words, tactile feedback may be reserved for a situation where the clinician is operating scroll wheel 122 to increase/decrease the stimulation parameter when a maximum/minimum threshold for the stimulation parameter has already been reached. Accordingly, tactile feedback may be used to indicate to the clinician that the maximum/minimum threshold for the stimulation parameter has been reached.

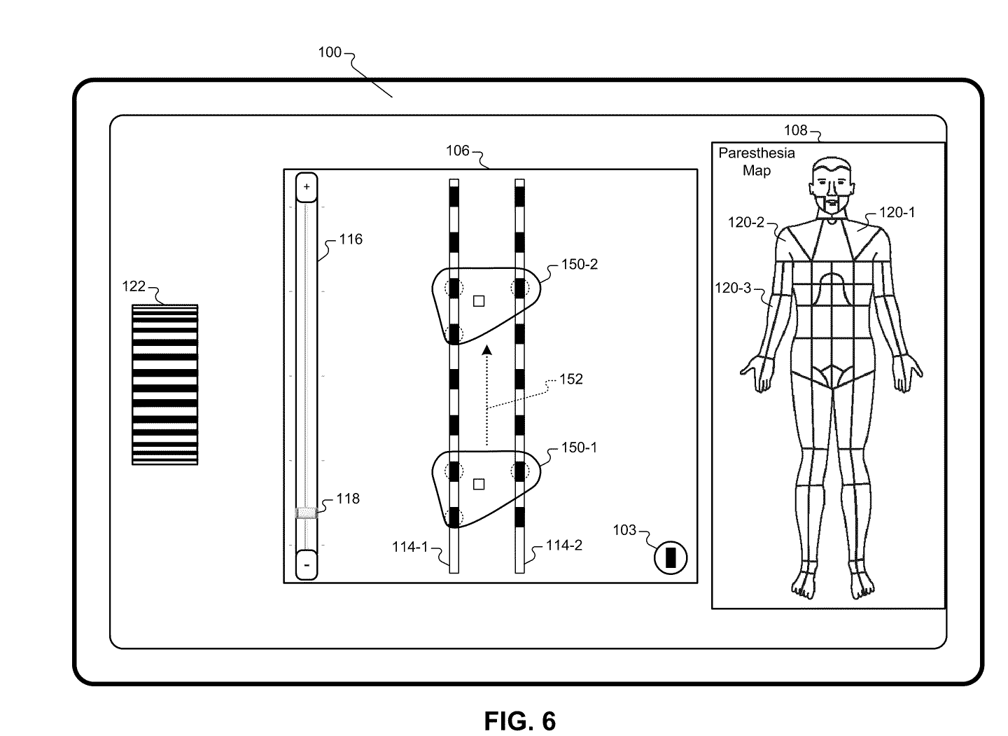

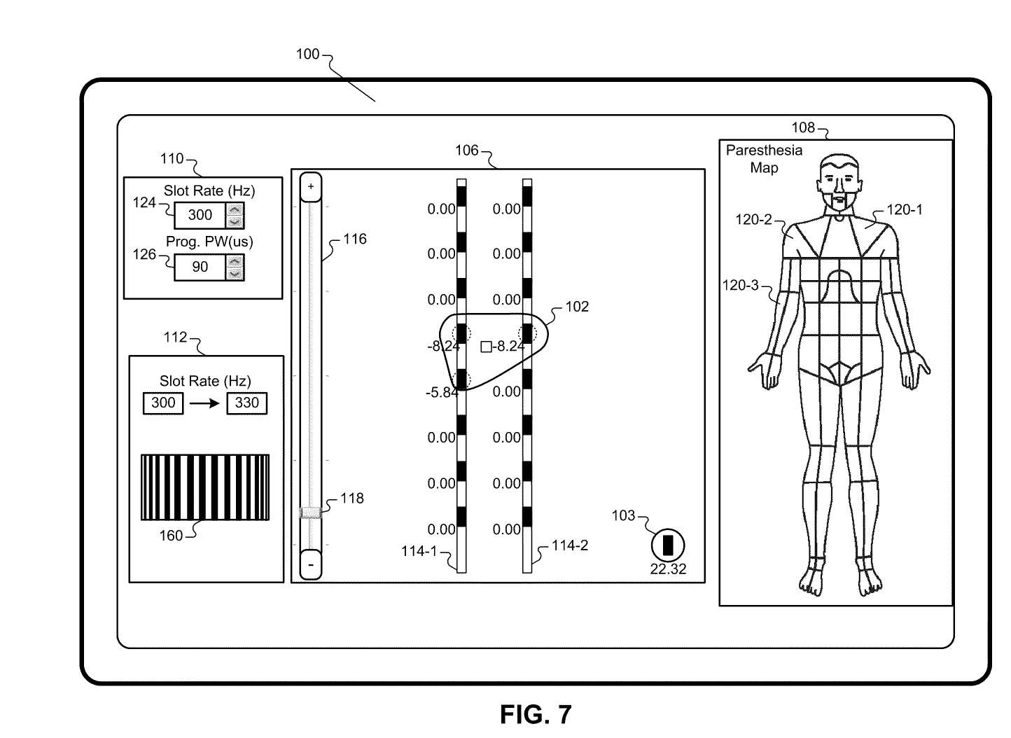

Referring now to FIG. 6, in addition to adjusting numeric stimulation parameters such as pulse rate, pulse width, and amplitude, scroll wheel 122 may be used to adjust an electrode configuration, i.e., a position of control shape 102 along electrodes 114-1 and 114-2. For example, scroll wheel 122 may be actuated in an upward/downward direction to adjust control shape 102 up/down the representation of leads 114-1 and 114-2, and accordingly adjust the stimulation region in patient 112 up and down electrodes 22 on leads 16. As shown in FIG. 6, a control shape may be moved up the representation of the leads 114-1 and 114-2. For example, FIG. 6 illustrates a control shape moving up the representation of leads 114-1 and 114-2 from a first position, illustrated at 150-1, to a second position, illustrated at 150-2. Direction of movement of the control shape is illustrated by the dotted arrow 152. Although movement of a control shape is illustrated for a two lead system, movement of a control shape in systems including more or less leads is contemplated. Although movement of a control shape up and down leads using a vertical scroll wheel is shown in FIG. 6, in other examples, a horizontal scroll wheel (e.g., scroll wheel 160 of FIG. 7) may be used to move a control shape up and down leads. In still other examples, a horizontal scroll wheel (e.g., scroll wheel 160) may be used to move a control shape horizontally (e.g., left/right) between leads. In examples that include both a vertical scroll wheel and a horizontal scroll wheel, the vertical and horizontal scroll wheels may be used to move a control shape up/down and left/right, respectively, along the leads.

In some implementations, the clinician may set maximum and minimum thresholds for movement of the control shapes. For example, the clinician may set a minimum threshold corresponding to how far the control shape may be moved toward a proximal end (e.g., near the stimulator 14) of leads 114-1 and 114-2. The clinician may also set a maximum threshold corresponding to how far the control shape may be moved toward a distal end of leads 114-1 and 114-2. With minimum and maximum thresholds set for the position of the control shape along leads 114-1 and 114-2, the clinician may adjust the field using scroll wheel 122 while observing patient 12, assured that the control shape will not move beyond the boundaries set by the minimum and maximum thresholds.

Referring back to FIG. 5, a GUI is shown in which the clinician has selected a pulse width stimulation parameter to control using scroll wheel 122. In the example GUI of FIG. 5, scrolling scroll wheel 122 upward may increase the value of the pulse width, while scrolling scroll wheel 122 downward may decrease the value of the pulse width. In other words, the clinician may swipe a finger from the bottom of scroll wheel 122 to the top of scroll wheel 122 to increase the value of the pulse width, and swipe their finger from the top of scroll wheel 122 to the bottom of scroll wheel 122 to decrease the value of the pulse width. The minimum and maximum thresholds illustrated for the pulse width are 80 and 100 μs, respectively. Accordingly, the clinician may only adjust the pulse width from the current value of 90 μs to a minimum of 80 μs and a maximum of 100 μs.

Although, FIGS. 4-5 show modification of pulse rate and pulse width, respectively, the clinician may select other stimulation parameters that may be controlled using scroll wheel 122. For example, the clinician may select pulse amplitude, and subsequently adjust pulse amplitude using scroll wheel 122.

FIG. 7 illustrates a GUI in which a scroll wheel 160 is oriented in a horizontal direction. Accordingly, the clinician may swipe their finger horizontally across touchscreen 66 to actuate scroll wheel 160. In the example GUI of FIG. 7, scrolling scroll wheel 160 toward the right may increase the value of the slot rate, while scrolling scroll wheel 160 to the left may decrease the value of the slot rate. The clinician may use the horizontal scroll wheel shown in FIG. 7 to adjust other stimulation parameters, such as the pulse width, amplitude, etc., in a manner similar to that in the GUI of FIG. 4.

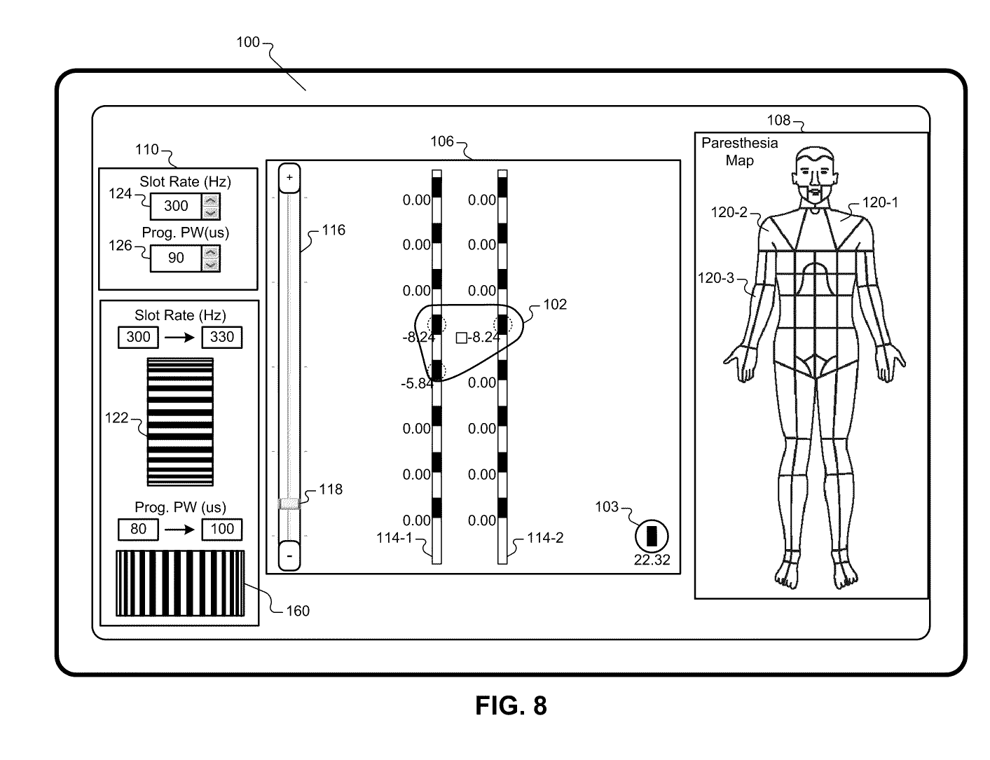

FIG. 8 illustrates a GUI that includes multiple scroll wheels 122 and 160. Scroll wheels 122 and 160 are each associated with separate stimulation parameters and corresponding minimum/maximum thresholds. For example, scroll wheel 122 may be used to adjust the pulse rate, while scroll wheel 160 may be used to adjust the pulse width. Each of scroll wheels 122 and 160 may be reassigned to different stimulation parameters. Accordingly, either of scroll wheels 122 or 160 may be assigned to modify pulse amplitude, pulse rate, pulse width, and electrode configuration.

In some implementations, each of scroll wheels 122 and 160, and accordingly each of the parameters, may be assigned different audible and/or tactile feedback parameters. Accordingly, the clinician may determine which of scroll wheels 122 and 160 they are interacting with, based on the different audible and/or tactile feedback, without looking back at display 64 of programmer 20. Different audible/tactile feedback parameters may include different tones associated with each of scroll wheels 122 and 160 and/or different frequencies of vibration associated with each of scroll wheels 122 and 160. For example, audible beeps associated with the clinician touching scroll wheel 122 may differ (e.g., in frequency content) from audible beeps associated with the clinician touching scroll wheel 160. As a further example, vibrations associated with the clinician touching scroll wheel 122 may differ (e.g., in frequency content) from vibrations associated with the clinician touching scroll wheel 160. Additionally, audible feedback may also indicate which of scroll wheels 122 or 160 is being rotated, and in which direction. For example, different tones may be associated with adjustment of each of scroll wheels 122 and 160. The different tones may vary depending on whether the adjustment is associated with an increase in the selected parameter or a decrease in the selected parameter. Specifically, in one implementation, the tones associated with each scroll wheel 122 and 160 may increase/decrease in frequency when the selected parameter is increased/decreased.

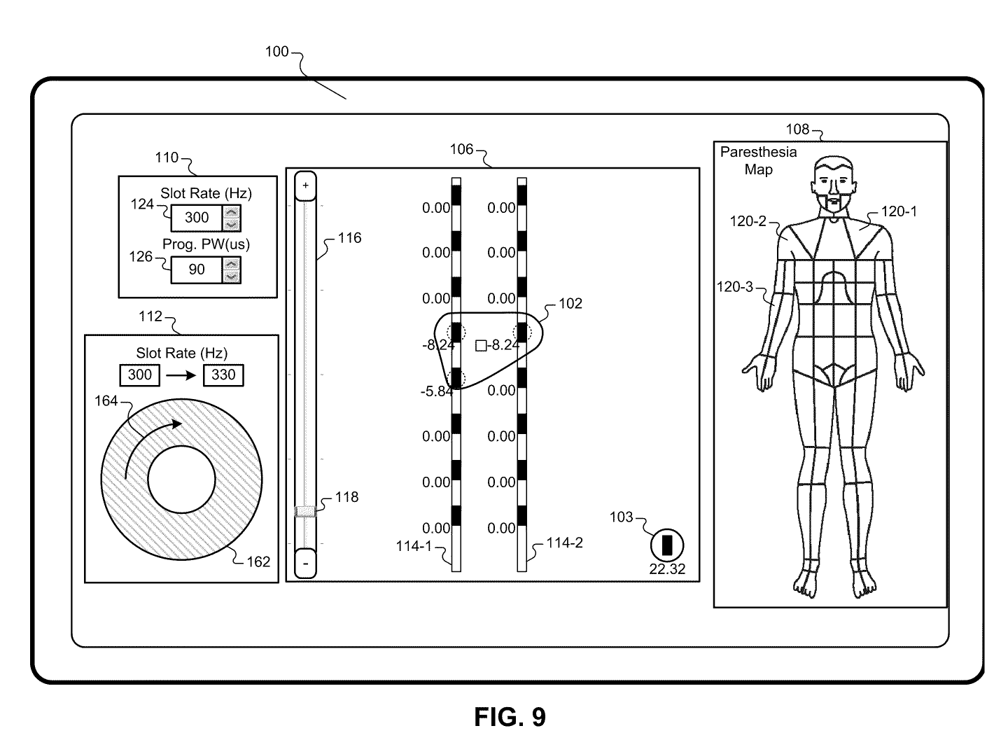

FIG. 9 shows an alternate control 162 displayed on display 64. Control 162 illustrated in FIG. 9 may be referred to as a “wheel control 162.” The clinician may actuate wheel control 162 by moving their finger in a clockwise or counter-clockwise, rotary direction around wheel control 162, e.g., tracing all or part of the shape of the wheel. An arrow 164 illustrates a clockwise direction of motion that may actuate wheel control 162. For example, actuating wheel control 162 in a clockwise direction may increase the value of the selected stimulation parameter, while actuating wheel control 162 in a counter-clockwise direction may decrease the value of the selected stimulation parameter. Accordingly, in FIG. 9, a clockwise actuation of wheel control 162 may increase the slot rate, while a counter-clockwise actuation of wheel control 162 may decrease the slot rate.

Wheel control 162 may include similar properties as scroll wheel 122. Wheel control 162 may be configured to operate based on various wheel control parameters. For example, the sensitivity of wheel control 162 may be adjusted. Wheel control 162 may include an inertia parameter that causes wheel control 162 to continue to rotate after wheel control 162 is actuated. Programmer 20 may provide feedback to the clinician while the clinician operates wheel control 162. Audible/tactile feedback associated with wheel control 162 may include sounds/vibrations that indicate whether the clinician's finger is touching wheel control 162, to what extent (i.e., a speed) wheel control 162 is being actuated, in which direction wheel control 162 is being rotated, and when wheel control 162 is being actuated to provide an adjustment that is prohibited by the minimum and maximum thresholds.

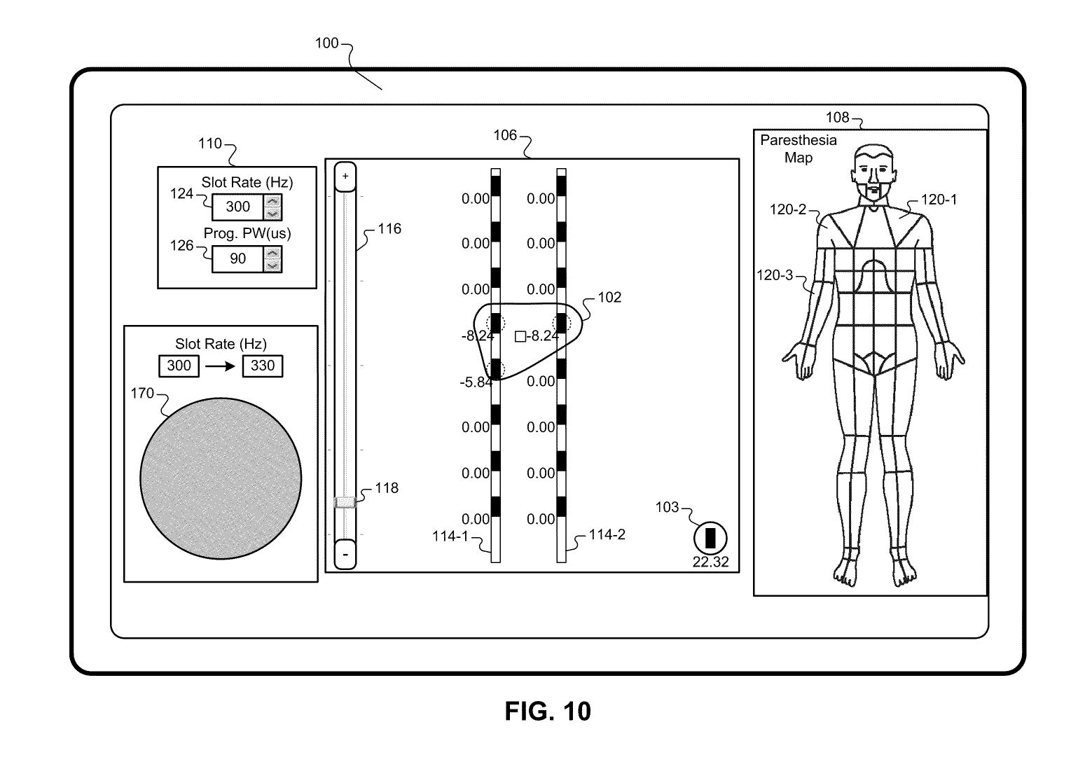

FIG. 10 shows an omni-directional control 170 displayed on display 64. The user may interact in a variety of ways with omni-directional control 170. In one implementation, touchscreen controller 54 may recognize linear gestures on omni-directional control 170, similar to those recognized on scroll wheels 122 and 160. Accordingly, a swiping gesture from the left side to the right side, or from the bottom to the top, of omni-directional control 170 may increase the selected stimulation parameter, while a swiping gesture from the right side to the left side, or from the top to the bottom, of omni-directional control 170 may decrease the selected stimulation parameter.

In other implementations, touchscreen controller 54 may recognize circular gestures on omni-directional control 170, similar to those recognized using wheel control 162. Accordingly, a circular gesture in a clockwise/counter-clockwise direction may increase/decrease the selected stimulation parameter.

Omni-directional control 170 may include similar properties as scroll wheels 122 and 160 and wheel control 162. Accordingly, omni-directional control 170 may be configured to operate based on various parameters. For example, the sensitivity of omni-directional control 170 may be adjusted. Omni-directional control 170 may include an inertia parameter that causes omni-directional control 170 to continue to adjust stimulation parameters after omni-directional control 170 is actuated. Programmer 20 may provide feedback to the clinician while the clinician operates omni-directional control 170. Audible/tactile feedback associated with omni-directional control 170 may include sounds/vibrations that indicate whether the clinician's finger is touching omni-directional control 170, to what extent (i.e., a speed) omni-directional control 170 is being actuated, in which direction omni-directional control 170 is being actuated, and when omni-directional control 170 is being actuated to provide an adjustment that is prohibited by the minimum and maximum thresholds.

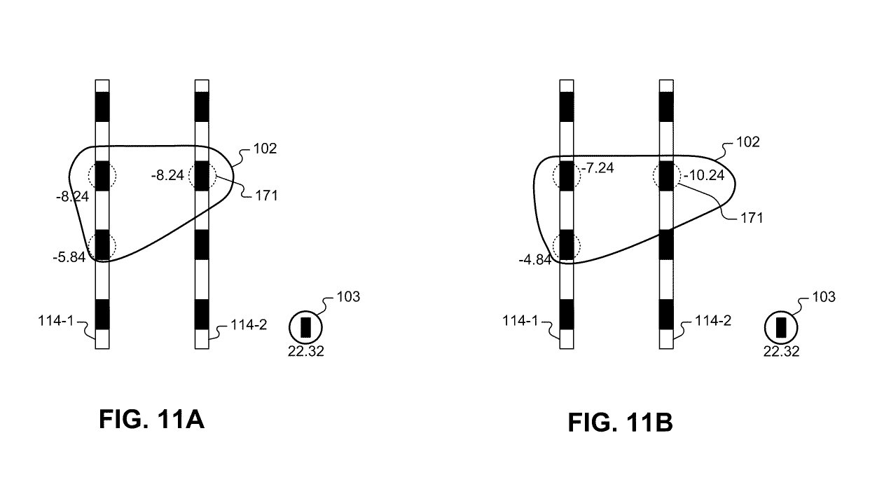

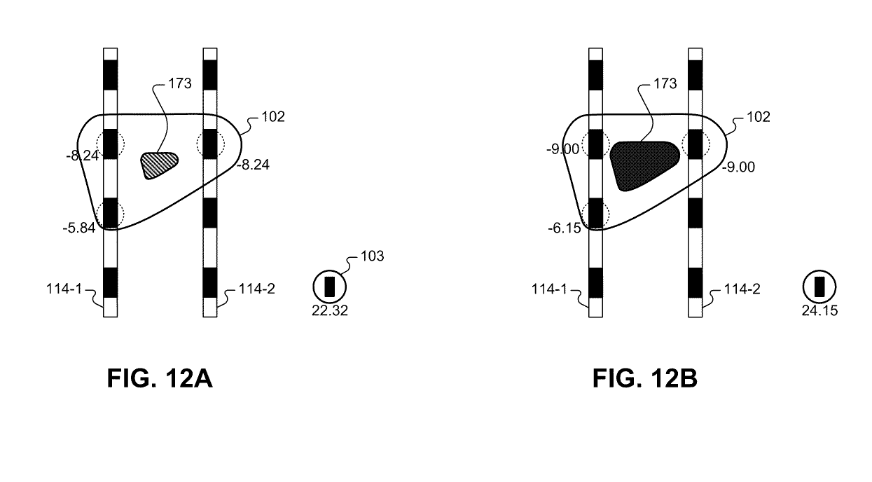

FIGS. 11A-11B and 12A-12B illustrate manipulation of control shape 102 (e.g., a cathodal control shape). FIGS. 11A-11B show how the shape and size of control shape 102 may be modified, for example, using the scroll wheel 122. In response to manipulation of the shape and/or size of control shape 102, programmer 20 may automatically adjust amplitude contributions of the electrodes that deliver stimulation. In some implementations, the clinician may click on an electrode and subsequently actuate scroll wheel 122 to manipulate the size/shape of control shape 102 using scroll wheel 122. For example, in FIG. 11A, the clinician may click on electrode 171, then actuate scroll wheel 122 upward/downward to increase/decrease the contribution of electrode 171 to a total amplitude of stimulation.

FIG. 11B illustrates an increase in amplitude at electrode 171 (e.g., −8.24 to −10.24) that may result from actuating scroll wheel 122 upward after selecting electrode 171 of FIG. 11A. The portion of control shape 102 associated with electrode 171 may expand after the amplitude of electrode 171 is set to −10.24. The amplitude associated with electrode 171 may subsequently be decreased back to −8.24 by actuating scroll wheel 122 in a direction opposite to that which caused the increase in amplitude (e.g., actuating scroll wheel 122 downward).

Although scroll wheel 122 is described as providing the functionality illustrated by FIGS. 11A-11B, other controls may also provide the functionality illustrated in FIGS. 11A-11B. For example, wheel control 162 and omni-directional control 170 may provide for adjustments of amplitude contributions of the electrodes. For example, actuation of wheel control 162 in a clockwise/counter-clockwise direction may result in an increase/decrease in amplitude associated with electrode 171. As a further example, swiping across omni-directional control 170 may result in manipulation of the amplitude associated with electrode 171.

Although adjustment of all amplitudes simultaneously is shown in FIGS. 11A-11B, in some examples, the amplitudes associated with each electrode may be adjusted individually without affecting amplitudes of other electrodes. Although scroll wheel 122 is described as adjusting amplitudes in FIGS. 11A and 11B, scroll wheel 122 may also be used to select whether an electrode acts as an anode, cathode, or is turned off. For example, an electrode may be selected and scroll wheel 122 may be actuated to cycle through a state of the electrode (i.e., on/off, anode, cathode) prior to adjusting amplitude associated with the electrode.

In some implementations, omni-directional control 170 may be used modify control shape 102 in an intuitive manner in order to adjust relative amplitude contributions of the electrodes. For example, swiping of omni-directional control 170 may correspond to manipulation of the shape of control shape 102 with respect to electrode 171. Specifically, in FIG. 11A, the user may select electrode 171, then swipe to the right on the omni-directional control 170 to expand distribution of control shape 102 around electrode 171 (e.g., from −8.24 to −10.24), as shown in FIG. 11B. Subsequently, the user may swipe to the left on omni-directional control 170 to restore the shape of control shape 102 to that of FIG. 11A.

Although FIGS. 11A and 11B illustrate that the change in control shape 102 may result in a change in relative amplitude contributions of each of the electrodes within control shape 102, in some implementations, changes in control shape 102 may result in changes in the number of electrodes recruited by control shape 102. For example, swiping downward on omni-directional control 170 while electrode 171 is selected may cause the electrode below electrode 171 to be recruited into control shape 102.

FIGS. 12A and 12B illustrate adjustment of the magnitude of all of the electrodes in control shape 102 simultaneously. An internal shape 173 may illustrate a combined amplitude of all of the electrodes in control shape 102 relative to a possible combined amplitude. For example, a smaller internal shape 173 (e.g., in FIG. 12A) may illustrate that the amplitude of all the electrodes may be increased, while a larger internal shape 173 (e.g., in FIG. 12B) may illustrate that there is less headroom to increase the amplitude of all the electrodes. The clinician may select internal shape 173 to adjust the size of internal shape 173, and accordingly the amplitude associated with control shape 102, using scroll wheel 122. For example, the clinician may select internal shape 173 of FIG. 12A and actuate scroll wheel 122 upwards to increase the combined amplitude of all of the electrodes in control shape 102. FIG. 12B illustrates the increase in combined amplitude of all of the electrodes relative to FIG. 12A. The amplitudes are illustrated as being increased from (−8.24, −8.24, −5.84) in FIG. 12A to (−9.00, −9.00,−6.15) in FIG. 12B. The clinician may subsequently actuate scroll wheel 122 in the opposite direction (e.g., downward) to decrease the amplitudes of FIG. 12B back to the amplitudes of FIG. 12A. Although scroll wheel 122 is described as providing the change in amplitudes of FIGS. 12A-12B, other controls may also provide the functionality of FIGS. 12A-12B. For example, wheel control 162 and omni-directional control 170 may provide for changes in amplitudes.

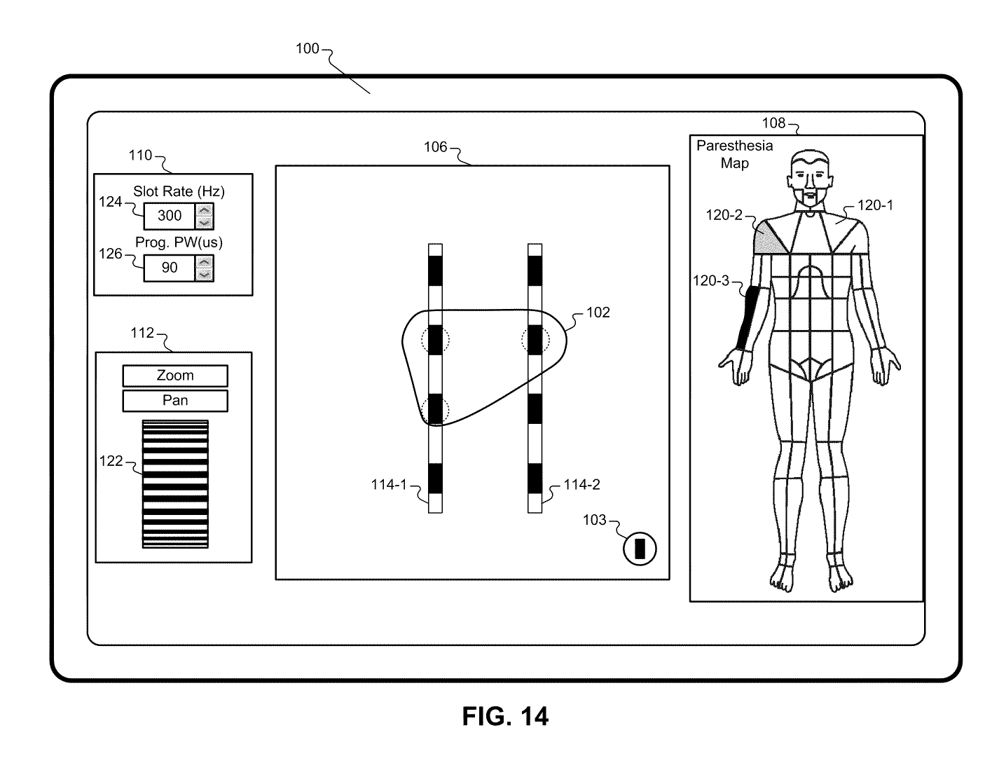

FIGS. 13-14 illustrate additional functionality of scroll wheel 122 that may be implemented in the GUI in addition to the control of stimulation parameters. FIG. 13 illustrates the use of scroll wheel 122 to interact with paresthesia map 108. The clinician may select regions of paresthesia map 108 and darken the regions to indicate an amount of paresthesia/pain felt by patient 12. The clinician may select the “select region” box in order to cycle through the regions (e.g., 120-1, 120-2, and 120-3) on the diagram using scroll wheel 122. For example, the clinician may actuate scroll wheel 122 up/down to cycle through the regions. Once the clinician has selected the appropriate region, the clinician may select the “fill region” box to darken the region using scroll wheel 122. For example, the clinician may actuate scroll wheel 122 up/down in order to darken/lighten the region. A darker/lighter region may indicate a greater/lesser amount of pain or paresthesia felt by patient 12. In some implementations, the clinician may select a color to use to darken the region. For example, a green may be used to indicate paresthesia, while a red may be used to indicate pain. Accordingly, a dark/light green may indicate a greater/lesser amount of paresthesia, while a dark/light red may indicate a greater/lesser amount of pain. In paresthesia map 108 of FIG. 13, region 120-1 includes no indication of paresthesia/pain since region 120-1 is not colored. Regions 120-2 and 120-3 have been shaded, and accordingly, may indicate an amount of paresthesia/pain. Region 120-3 is shaded darker than region 120-2, and accordingly region 120-3 may indicate a greater amount of paresthesia/pain, depending on the color of the regions.

FIG. 14 illustrates using scroll wheel 122 to perform a zoom function. In some implementations, the clinician may zoom in/out on the representations of leads 114-1 and 114-2 using scroll wheel 122. For example, scroll wheel 122 may be scrolled up/down to zoom in/out on the representations of leads 114-1 and 114-2. Lead display window 106 in FIG. 14 illustrates a view of leads 114-1 and 114-2 that is zoomed in relative to that shown in FIGS. 4-10, 13, 15, and 16. As discussed above, slider 118 of sliding control 116 of FIGS. 4-10, 13, and 15-16 may also be used to zoom in on leads 114-1 and 114-2. Accordingly, both scroll wheel 122 and slider control 116 may be used to zoom in on leads 114-1 and 114-2. In some implementations, actuation of scroll wheel 122 may allow the user to pan up and down leads 114-1 and 114-2. For example, the user may select the “pan” box or the “zoom” box to switch between zooming to panning.

Although scroll wheel 122 is illustrated as providing the functionality of FIGS. 13-14, other controls may also provide the functionality of FIGS. 13-14. For example, wheel control 162 and omni-directional control 170 may provide for interaction with paresthesia map 108 and may also provide the zooming/panning function.

For example, the clockwise/counter-clockwise rotation of wheel control 162 may cycle through and darken/lighten the regions on paresthesia map 108. Clockwise/counter-clockwise rotation of wheel control 162 may also allow for zooming in/out on the representation of leads 114-1 and 114-2. Similarly, swiping gestures and rotational gestures performed on omni-directional control 170 may allow for cycling through paresthesia map 108, darkening/lightening regions of paresthesia map 108, and zooming in/out on leads 114-1 and 114-2. In some implementations, omni-directional control 170 may allow for support of multi-touch control. Accordingly, zooming in/out may be performed on omni-directional control 170 via a pinch and zoom operation. For example, the clinician may spread their fingers on omni-directional control 170 to zoom into leads 114-1 and 114-2, and pinch their fingers together on omni-directional control 170 to zoom out from leads 114-1 and 114-2.

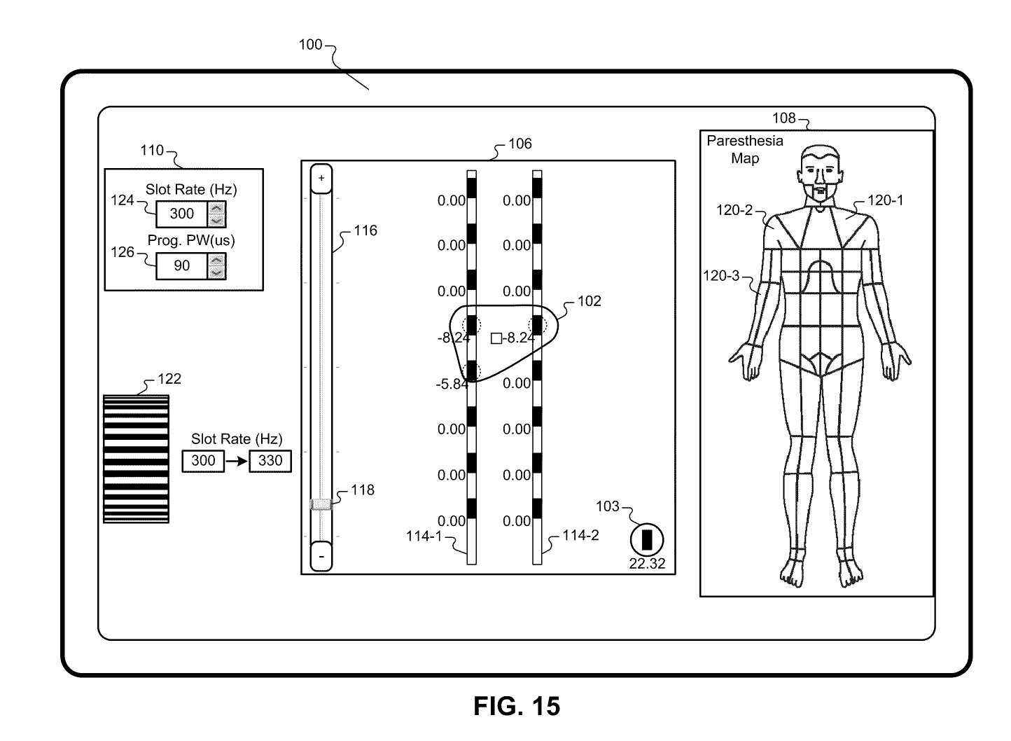

FIG. 15 shows how placement of scroll wheel 122 on display 64 may aid the clinician in operating programmer 20 without looking at display 64. Selective placement of scroll wheel 122 on display 64 may allow the clinician to interact with patient 12, without focusing on display 64 to control scroll wheel 122. Specifically, FIG. 15 illustrates that scroll wheel 122 may be displayed adjacent to bezel 100 surrounding display 64. More specifically, scroll wheel 122 may be arranged so that the clinician may simultaneously contact both bezel 100 and the region of display 64 that includes scroll wheel 122 using their finger. In other words, placement of scroll wheel 122 adjacent to bezel 100 may readily allow the clinician to operate scroll wheel 122 without looking at display 64 since the clinician may determine a position of scroll wheel 122 based on the position of bezel 100.

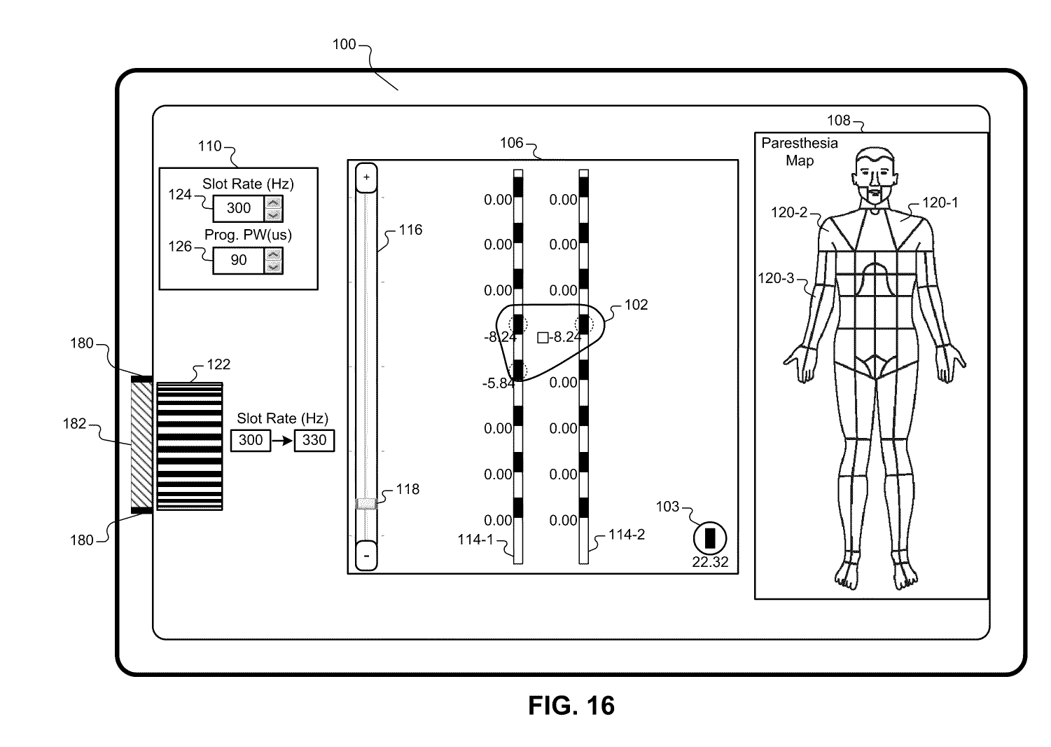

FIG. 16 shows a modification to bezel 100 that may further allow the clinician to operate scroll wheel 122 without looking at display 64. Bezel 100 of FIG. 16 includes surface features that indicate a position of scroll wheel 122 along bezel 100. For example, the surface features may include raised edges 180 or a textured region 182. In some implementations, raised edges 180 and textured region 182 may be replaced or complemented by surface features such as recessed regions, dimpled regions, ridged regions, and/or knurled regions, for example. Raised edges 180 on bezel 100 may indicate to the clinician, based on sense of touch, where the edges of scroll wheel 122 are located. Additionally, textured region 182 of bezel 100 may indicate to the clinician, based on touch, where along bezel 100 scroll wheel 122 is located. Accordingly, the clinician may, based on sensing a texture or feature of bezel 100, determine a location of scroll wheel 122 along bezel 100. Therefore, surface features on bezel 100 may improve the clinician's ability to locate scroll wheel 122 on display 64 without viewing display 64.

Location of scroll wheel 122 and other controls on the left side of display 64 may be beneficial for right-handed clinicians, since the clinician may use a stylus to interact with programmer 20 in their right hand while operating scroll wheel 122 with their left hand. Although scroll wheel 122, wheel control 162, and omni-directional control 170 are illustrated on the left side of display 64 in FIGS. 4-10 and 13-16, scroll wheel 122, wheel control 162, and omni-directional control 170 may be located at other locations on the display, depending on the layout of the GUI.

In some implementations, the user may specify the location of scroll wheel 122 on display 64, e.g., via a user setup menu. For example, the user may specify whether scroll wheel 122 is on the left or right side of display 64. In some examples, the user may specify any location on display 64 for scroll wheel 122 using the user setup menu. Using the user setup menu, the user may also specify other adjustments to the GUI. For example, the user may select whether the GUI is displayed in a portrait or landscape mode. User may then further specify the location of scroll wheel 122 within the portrait or landscape GUI using the user setup menu.

Although programmer 20 is described as including touchscreen 66 that presents a graphical, gesture-based input medium, such as a graphical scroll wheel, programmer 20 may also be connected to other input devices that may be used by the clinician to adjust one or more medical device parameters. For example, programmer 20 may include a universal serial bus (USB), or other suitable peripheral bus, that allows for connection of programmer 20 to a mechanical input device. Accordingly, the clinician may connect a mechanical input device to programmer 20 for adjusting one or more medical device parameters.

A mechanical input device may include a device which is mechanically actuated by the clinician, such as a mechanical scroll wheel or a trackball, for example. The mechanical input device may operate programmer 20 in a similar fashion as the graphical scroll wheel, the graphical rotary control wheel, and the graphical omni-directional touch pad as described herein. For example, the user may select a stimulation parameter, and then adjust the parameter by actuating the mechanical device. As a further example, the user may select control shape 102, and then modify the shape, size, and position of control shape 102 by actuating the mechanical device. Accordingly, in some examples, the clinician may use the mechanical device in conjunction with the touchscreen 66 to adjust one or more medical device parameters.

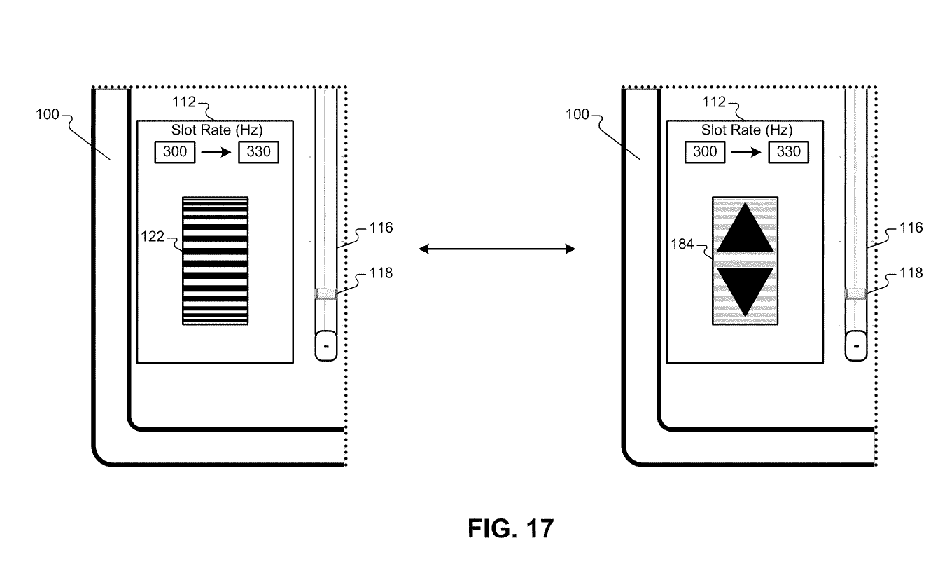

Referring now to FIG. 17, the user may interact with touchscreen 66 to transition from a first type of control to a second type of control. FIG. 17 illustrates a transition between scroll wheel 122 and a discrete control 184. In one example, the user may transition from scroll wheel 122 to discrete control 184 by pressing down on scroll wheel 122 for a predetermined amount of time (e.g., a few seconds). In other words, the user may transition from scroll wheel 122 to discrete control 184 by pressing and holding scroll wheel 122 for the predetermined amount of time without swiping across scroll wheel 122. The user may transition back to scroll wheel 122 by tapping on touchscreen 66 in a location other than where discrete control 184 is located, i.e., anywhere on touchscreen 66 other than on discrete control 184.

The graphic representing scroll wheel 122 may transition to the graphic representing discrete control 184 after the user presses and holds scroll wheel 122 for the predetermined amount of time. The graphic representing discrete control 184, as illustrated in FIG. 17, includes two darkened triangles overlaying a lightened image of the scroll wheel graphic. The user may actuate discrete control 184 by tapping on one of the darkened triangles. A tap on the darkened triangles may provide discrete changes in the selected parameter. For example, a tap on the upward pointing triangle may increase the selected parameter (i.e., slot rate) by a discrete amount, while a tap on the downward pointing triangle may decrease the selected parameter by a discrete amount. The discrete amount may be selectable by the user. In one example, the discrete amount may be a minimum amount by which the selected parameter may be adjusted. Accordingly, discrete control 184 may be used to make minimal discrete adjustments to the selected parameter. In other words, discrete control 184 may be used to finely adjust the selected parameter.

Although switching from scroll wheel 122 to discrete control 184 is described, switching between any type of control may be implemented. For example, the user may press and hold any of the other controls described herein (i.e., wheel control 162, omni-directional control 170) to transition to discrete control 184. In other examples, the user may transition from any control described herein to any other control described herein by pressing and holding for the predetermined amount of time.

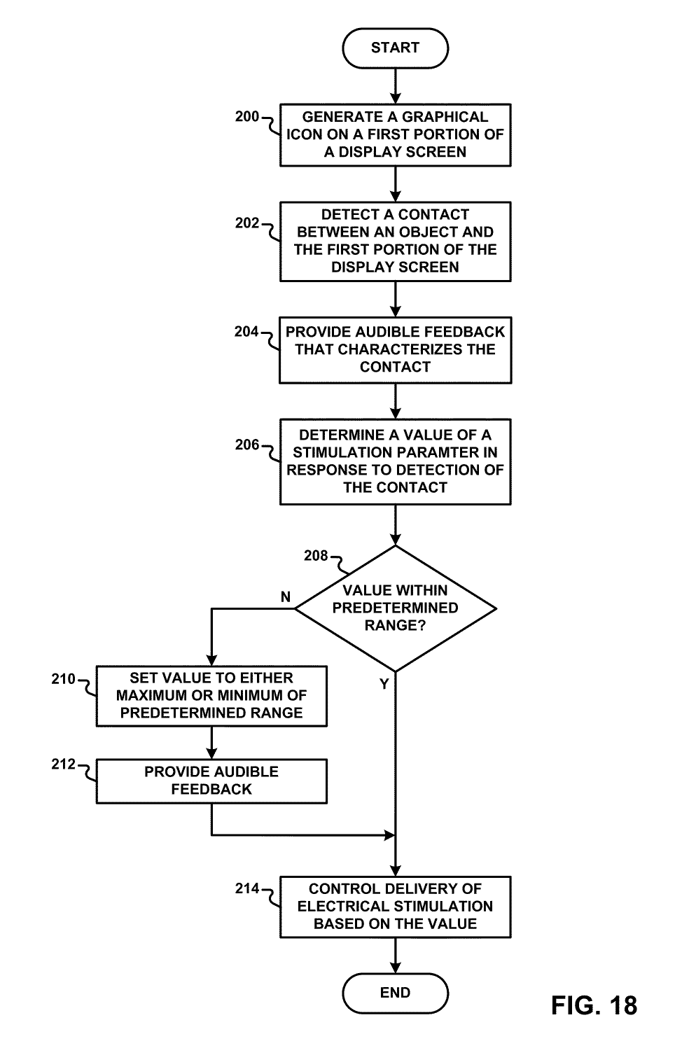

FIG. 18 is a flow diagram illustrating a method for communicating with an implantable electrical stimulator using a programmer. As shown in FIG. 18, display controller 52 generates a graphical icon on a first portion of display 64 (200). Touchscreen controller 54 detects a contact between an object (e.g., a finger) and the first portion of display 64 (202). Feedback device 70 provides audible feedback that characterizes the contact (204). For example, the audible feedback may indicate whether a finger is touching the control icon (e.g., scroll wheel 122), to what extent (i.e., a speed) the control icon is being actuated, and in which direction the control icon is being actuated.

Processor 56 determines a value of a stimulation parameter in response to the detection of the contact (206). For example, the stimulation parameter may include at least one of a pulse amplitude, a pulse width, and a pulse rate. Processor 56 determines whether the value of the stimulation parameter is within a predetermined range set by the clinician (208). If the value is within the predetermined range, stimulator 14 provides stimulation using the value for the stimulation parameter (214).

If the value is not within the predetermined range, communication module 60 sets the value of the stimulation parameter in stimulator 14 to a threshold value (210) and feedback device 70 indicates that the value is not within the predetermined range (212). For example, if the value is equal to or greater than the maximum of the predetermined range, the communication module 60 sets the value to the maximum of the predetermined range. Alternatively, if the value is equal to or less than the minimum of the predetermined range, communication module 60 sets the value to the minimum of the predetermined range.

The techniques described in this disclosure may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, stimulators, or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as RAM, ROM, NVRAM, EEPROM, FLASH memory, magnetic data storage media, optical data storage media, or the like. The instructions may be executed to support one or more aspects of the functionality described in this disclosure.

Many embodiments of the disclosure have been described. Various modifications may be made without departing from the scope of the claims. These and other embodiments are within the scope of the following claims.

1. A programming device comprising:
a touchscreen display;
a processor that controls the display to present a graphical icon on a first portion of the display, that detects a gesture-based contact between an object and the first portion of the display, and that determines a value of a therapy parameter associated with therapy delivered by a medical device based on the detection of the gesture-based contact; and
a communication module that transmits information to the medical device to control the medical device to deliver the therapy based on the value of the therapy parameter.

2. The programming device of claim 1, wherein the graphical icon represents a control that is configured to receive a gesture-based input from a user.

3. The programming device of claim 2, wherein the control comprises a graphical representation of a scroll wheel.

4. The programming device of claim 1, wherein the gesture-based contact includes a swipe of the object across the graphical icon presented on the display.

5. The programming device of claim 4, wherein the processor determines the value by incrementing a current value of the therapy parameter when the swipe is in a first direction, and wherein the processor determines the value by decrementing the current value when the swipe is in a direction that is opposite to the first direction.

6. The programming device of claim 1, wherein the therapy parameter indicates at least one of a duration of an electrical pulse, a number of electrical pulses per unit of time, and a magnitude of an electrical pulse.

7. The programming device of claim 1, wherein the processor determines a direction of a path that is traversed by the object while the object is in contact with the first portion, and wherein the processor determines the value based on the direction of the path.

8. The programming device of claim 7, wherein the processor increments the value when the direction of the path is in a first direction, and wherein the processor decrements the value when the direction of the path is in a direction that is opposite to the first direction.

9. The programming device of claim 1, wherein the processor determines a speed of the object while the object is in contact with the first portion, and wherein the processor determines the value based on the speed of the object.

10. The programming device of claim 9, wherein the processor determines an amount to increment the value based on the speed of the object.

11. The programming device of claim 1, further comprising a feedback device that generates audible feedback to a user of the programming device in response to the contact between the object and the first portion of the display.

12. The programming device of claim 11, wherein the feedback device generates audible feedback that indicates when the value determined by the processor is equal to or greater than a maximum threshold of a predetermined range of values set by the user.

13. The programming device of claim 11, wherein the processor determines a direction of a path that is traversed by the object while the object is in contact with the first portion, and wherein the feedback device generates audible feedback that indicates the direction.

14. The programming device of claim 11, wherein the processor determines a speed of the object while the object is in contact with the first portion, and wherein the feedback device generates audible feedback that indicates the speed of the object.

15. The programming device of claim 1, wherein the object includes one of a finger and a stylus.

16. The programming device of claim 1, wherein the touchscreen display includes one of a resistive touchscreen device and a capacitive touchscreen device, and wherein the processor communicates with the one of the resistive touchscreen device and the capacitive touchscreen device.

17. The programming device of claim 1, wherein the medical device includes an implantable electrical stimulator (IES), and wherein the communication module sets an electrode configuration of the IES in response to the contact.

18. The programming device of claim 1, wherein the processor determines whether the value is within a predetermined range of values set by a user, and wherein the communication module sets the therapy parameter to the value when the value is within the predetermined range of values.

19. The programming device of claim 1, further comprising a feedback device that generates haptic feedback to a user of the programming device in response to the contact between the object and the first portion of the display.

20. The programming device of claim 1, further comprising a bezel formed around the touchscreen display, wherein the processor controls the display to present the graphical icon along an edge of the bezel.

21. The programming device of claim 20, wherein a portion of the bezel located adjacent to the graphical icon includes a texturing that indicates a location of the graphical icon.

22. The programming device of claim 2, wherein the graphical icon that represents the control is configured to receive input from the user that transitions the graphical icon to a different graphical icon that represents a different control that is configured to receive a different gesture-based input from the user.

23. A method comprising:
presenting a graphical icon on a first portion of a touchscreen display;
detecting a gesture-based contact between an object and the first portion of the display;
determining a value of a therapy parameter associated with therapy delivered by a medical device based on the detection of the gesture-based contact; and
transmitting information to the medical device to control the medical device to deliver the therapy based on the value of the therapy parameter.

24. The method of claim 23, wherein the gesture-based contact includes a swipe of the object across the graphical icon presented on the display.

25. The method of claim 24, further comprising:
determining the value by incrementing a current value of the therapy parameter when the swipe is in a first direction; and
determining the value by decrementing the current value when the swipe is in a direction that is opposite to the first direction.

26. The method of claim 23, further comprising:
determining a direction of a path that is traversed by the object while the object is in contact with the first portion; and
determining the value based on the direction of the path.

27. The method of claim 23, further comprising:
determining a speed of the object while the object is in contact with the first portion; and
determining the value based on the speed of the object.

28. The method of claim 23, further comprising generating audible feedback to a user in response to the contact between the object and the first portion of the display.

29. The method of claim 23, further comprising:
determining a direction of a path that is traversed by the object while the object is in contact with the first portion; and
generating audible feedback that indicates the direction.

30. The method of claim 23, further comprising:
determining whether the value is within a predetermined range of values set by a user; and
setting the therapy parameter to the value when the value is within the predetermined range of values.

31. A system comprising:
a medical device; and
a programming device comprising:
a touchscreen display;
a processor that controls the display to present a graphical icon on a first portion of the display, that detects a gesture-based contact between an object and the first portion of the display, and that determines a value of a therapy parameter associated with therapy delivered by the medical device based on the detection of the gesture-based contact; and
a communication module that transmits information to the medical device to control the medical device to deliver the therapy based on the value of the therapy parameter.

32. The system of claim 31, wherein the graphical icon represents a control that is configured to receive a gesture-based input from a user.

33. The system of claim 32, wherein the control comprises a graphical representation of a scroll wheel.

34. The system of claim 31, wherein the gesture-based contact includes a swipe of the object across the graphical icon presented on the display.

35. The system of claim 31, wherein the medical device includes an implantable electrical stimulator (IES), and wherein the therapy parameter indicates at least one of a duration of an electrical pulse, a number of electrical pulses per unit of time, a magnitude of an electrical pulse, and an electrode configuration.

36. The system of claim 31, wherein the processor determines a direction of a path that is traversed by the object while the object is in contact with the first portion, and wherein the processor determines the value based on the direction of the path.

37. The system of claim 31, further comprising a feedback device that generates one of audible and haptic feedback to a user of the programming device in response to the contact between the object and the first portion of the display.
